(19) United States
(12) Patent Application Publication
(10) Pub. No.: US 2020/0216787 A1
(43) Pub. Date: Jul. 9, 2020

(54) RIBOFLAVINASE ENZYMES AND THEIR USE TO PREVENT OFF FLAVOR IN BREWING

(71) Applicant: DuPont Nutrition Biosciences ApS, Copenhagen K (DK)

(72) Inventors: Hong Yang, Newark, DE (US); Toh Mei Yan Joyce, Langhorne, PA (US); Jeremy Ondov, New York, NY (US); Aaron Venables, Yardley, PA (US); Despina Bougioukou, Wilmington, DE (US)

(21) Appl. No.: 16/648,349

(22) PCT Filed: Sep. 17, 2018
(86) PCT No.: PCT/US18/51379

§ 371 Date: Mar. 18, 2020

Related U.S. Application Data
(60) Provisional application No. 62/556,979, filed on Sep. 11, 2017.

(51) Int. Cl. C12H 1/00; C12N 9/78; C12N 9/06; C12N 9/02

(52) U.S. Cl. CPC C12H 1/003; C12N 9/78; C12Y 305/99001; C12Y 114/9904; C12N 9/0028; C12N 9/0083; C12Y 105/01041

(57) ABSTRACT

The present disclosure provides methods, compositions, apparatuses and kits comprising several enzymes involved in the hydrolysis or breakdown of riboflavin beer or a malt-based substrate for beverage production. The present invention relates to improvements in the production of beer and similar light-sensitive beverages, in particular improvement of the flavor stability of such light-sensitive beverages by enzymatic hydrolysis or degradation of riboflavin during beer production.

### BACKGROUND OF THE INVENTION

Exposure of beer to sun light can result in the formation of an off-flavor called skunked beer. Brewers refer to this phenomenon as light struck or sun struck. Formation of skunked flavor in beer is obviously highly undesirable. To prevent the negative interaction between sun light and beer, brewers use glass dark, brown bottles that partly limits transmission of the visible and UV region of the spectrum. While brown bottles can be used to inhibit development of skunk flavor, brewers frequently use clear or light green bottles for beer storage for reasons related marketing and product differentiation. Light struck remains a major challenge for beer stored in green or clear bottles.

It is known that the compound giving rise to skunk beer is 3MBT (3-methylbut-ene-thiol, aka “skunky thiol”). Mechanism for Formation of the Lightstruck Flavor in Beer Revealed by Time-Resolved Electron Paramagnetic Resonance, Burns et al. 2001. Chem. Eur. J. 7 (21): 4553-4561. Skunky thiol is formed by the ultraviolet light induced reaction of sulfur containing amino acids with iso-humulones. Formation of 3MBT requires the presence of a photosensitizer which produces free radicals.

There is a need to prevent or inhibit the formation of skunky thiol in beer.

### SUMMARY OF THE INVENTION

In accordance with an aspect of the present invention, a method is presented for the inhibition of formation of 3MBT (3-methylbut-ene-thiol) in a malt beverage having the step of adding to the malt beverage an effective amount of a riboflavinase enzyme.

Optionally, the riboflavinase is a riboflavin hydrolase. Optionally, the riboflavin hydrolase is an enzyme having at least 80% sequence identity to MOXRcaE1 (SEQ ID NO:8) or an active fragment thereof or MOXRcaE2 (SEQ ID NO: 12) or an active fragment thereof.

Optionally, the riboflavin hydrolase is an enzyme having at least 90%, 95%, or 99% amino acid sequence identity to MOXRcaE1 or an active fragment thereof. Optionally, the riboflavin hydrolase is MOXRcaE1 or an active fragment thereof.

Optionally, the riboflavin hydrolase is an enzyme having at least 90%, 95%, or 99% amino acid sequence identity to MOXRcaE2 or an active fragment thereof. Optionally, the riboflavin hydrolase is MOXRcaE2 or an active fragment thereof.

Optionally, the riboflavinase is a riboflavin destructase.

Optionally, the riboflavin destructase is an enzyme having at least 80%, 90%, 95%, 99% identity to SmeBluB1 (SEQ ID NO:2) or an active fragment thereof or PspBluB1 (SEQ ID NO:4) or an active fragment thereof.

Optionally, the riboflavin destructase is SmeBluB1 or an active fragment thereof or PspBluB1 or an active fragment thereof.

Optionally, in the method of preventing the formation of 3MBT a second riboflavinase is used in addition to the first riboflavinase.

Optionally, the second riboflavinase is a riboflavin reductase.

Optionally, the riboflavin reductase is an enzyme having at least 80% identity to MOXRcaB1 (SEQ ID NO: 6) or an active fragment thereof or MOXRcaB2 (SEQ ID NO:10) or an active fragment thereof.

Optionally, the riboflavin reductase is an enzyme having at least 90%, 95% or 99% amino acid sequence identity to MOXRcaB1. Optionally, the riboflavin reductase is MOXcaB1 or an active fragment thereof.

Optionally, the riboflavin reductase is an enzyme having at least 90%, 95% or 99% amino acid sequence identity to MOXRcaB2. Optionally, the riboflavin reductase is MOXcaB2 or an active fragment thereof.

Optionally, the malt beverage of the instant invention is selected from the group consisting of a beer, lager, ale, dry beer, near beer, light beer, low alcohol beer, low calorie beer, porter, bock beer, stout, malt liquor, and non-alcoholic malt liquor. Optionally, the malt beverage is a beer.

In accordance with another aspect of the present invention, a malt beverage is presented having an effective amount of a riboflavinase as described above.

### BRIEF DESCRIPTION OF THE DRAWINGS

FIG. 1 depicts a plasmid map of p3JM-PspBluB2.

FIG. 2 depicts a plasmid map of pET-28b-SmeBluB1.

FIG. 3 Relative quantified (Index value) of 3-MBT in a regular German pilsner style beer either canned or bottled, as indicated, after exposure to light in transparent flasks for 0, 3 and 5 hours. Results are shown with regard to two identical experiments: 1 and 2.

FIG. 4 HPLC chromatogram (Abs. 340 nm) of in vitro, enzymatic degradation of riboflavin (RF) by A) RF+FMN+NADH+MOXRcaB1+MOXRcaE1 and B) RF+FMN+NADH+MOXRcaB2+MOXRcaE2 after 10 (black) and 20 (red) minutes, as described in example 4.

FIG. 5 depicts relative quantified (Index value) 3-MBT results for beer samples; untreated (beer) and treated with riboflavin binding protein (beer+RfBP), after 0 and 4 hours light exposure.

FIG. 6 depicts riboflavin content in beer samples; untreated (beer) and treated with riboflavin binding protein (Beer+RfBP), after 0 and 4 hours light exposure.

### BRIEF DESCRIPTION OF SEQ ID NOS

SEQ ID NO:1 sets forth the nucleotide sequence of the full-length SmeBluB1 gene identified from NCBI database.

SEQ ID NO:2 sets forth the predicted amino acid sequence of SmeBluB1.

SEQ ID NO:3 sets forth the nucleotide sequence of the full-length PspBluB2 gene identified from NCBI database.

SEQ ID NO:4 sets forth the predicted amino acid sequence of PspBluB2.

SEQ ID NO:5 sets forth the nucleotide sequence of the full-length MoxRcaB1 gene identified from NCBI database.

SEQ ID NO:6 sets forth the predicted amino acid sequence of MoxRcaB1.

SEQ ID NO:7 sets forth the nucleotide sequence of the full-length MoxRcaE1 gene identified from NCBI database.

SEQ ID NO:8 sets forth the predicted amino acid sequence of MoxRcaE1.

SEQ ID NO:9 sets forth the nucleotide sequence of the full-length MoxRcaB2 gene identified from NCBI database.

SEQ ID NO:10 sets forth the predicted amino acid sequence of MoxRcaB2.

SEQ ID NO:11 sets forth the nucleotide sequence of the full-length MoxRcaE2 gene identified from NCBI database.

SEQ ID NO:12 sets forth the predicted amino acid sequence of MoxRcaE2.

SEQ ID NO:13 sets forth the nucleotide sequence of the synthesized PspBluB2 gene in plasmid p3JM-PspBluB2.

SEQ ID NO:14 sets forth the nucleotide sequence of the synthesized MoxRcaE1 gene in plasmid p3JM-MoxRcaE1.

SEQ ID NO:15 sets forth the nucleotide sequence of the synthesized MoxRcaE2 gene in plasmid p3JM-MoxRcaE2.

SEQ ID NO:16 sets forth the nucleotide sequence of the synthesized SmeBluB1 gene in plasmid pET-28b-SmeBluB1.

SEQ ID NO:17 sets forth the nucleotide sequence of the synthesized MoxRcaB1 gene in plasmid pET-28b-MoxRcaB1.

SEQ ID NO:18 sets forth the nucleotide sequence of the synthesized MoxRcaB2 gene in plasmid pET-28b-MoxRcaB2.

SEQ ID NO:19 sets forth the amino acid sequence of SmeBluB1 expressed from plasmid pET-28b-SmeBluB1. The thrombin cleavage peptide was showed in bold and the 6×His-tag was showed in italics.

SEQ ID NO:20 sets forth the amino acid sequence of MoxRcaB1 expressed from plasmid pET-28b-MoxRcaB1. The thrombin cleavage peptide was showed in bold and the 6×His-tag was showed in italics.

SEQ ID NO.21 sets forth the amino acid sequence of MoxRcaB2 expressed from plasmid pET-28b-MoxRcaB2. The thrombin cleavage peptide was showed in bold and the 6×His-tag was showed in italics.

### DETAILED DESCRIPTION OF THE INVENTION

The practice of the present teachings will employ, unless otherwise indicated, conventional techniques of molecular biology (including recombinant techniques), microbiology, cell biology, and biochemistry, which are within the skill of the art. Such techniques are explained fully in the literature, for example, Molecular Cloning: A Laboratory Manual, second edition (Sambrook et al., 1989); Oligonucleotide Synthesis (M. J. Gait, ed., 1984; Current Protocols in Molecular Biology (F. M. Ausubel et al., eds., 1994); PCR: The Polymerase Chain Reaction (Mullis et al., eds., 1994); Gene Transfer and Expression: A Laboratory Manual (Kriegler, 1990), and The Alcohol Textbook (Ingledew et al., eds., Fifth Edition, 2009), and Essentials of Carbohydrate Chemistry and Biochemistry (Lindhorste, 2007).

Unless defined otherwise herein, all technical and scientific terms used herein have the same meaning as commonly understood by one of ordinary skill in the art to which the present teachings belong. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this invention. Any methods and materials similar or equivalent to those described herein can be used in the practice or testing of the present teachings.

Numeric ranges provided herein are inclusive of the numbers defining the range.

### Definitions and Abbreviations

The terms, “wild-type,” “parental,” or “reference,” with respect to a polypeptide, refer to a naturally-occurring polypeptide that does not include a man-made substitution, insertion, or deletion at one or more amino acid positions. Similarly, the terms “wild-type,” “parental,” or “reference,” with respect to a polynucleotide, refer to a naturally-occurring polynucleotide that does not include a man-made nucleoside change. However, note that a polynucleotide encoding a wild-type, parental, or reference polypeptide is not limited to a naturally-occurring polynucleotide, and encompasses any polynucleotide encoding the wild-type, parental, or reference polypeptide.

Reference to the wild-type polypeptide is understood to include the mature form of the polypeptide. A “mature” polypeptide or variant, thereof, is one in which a signal sequence is absent, for example, cleaved from an immature form of the polypeptide during or following expression of the polypeptide.

The term “variant,” with respect to a polypeptide, refers to a polypeptide that differs from a specified wild-type, parental, or reference polypeptide in that it includes one or more naturally-occurring or man-made substitutions, insertions, or deletions of an amino acid. Similarly, the term “variant,” with respect to a polynucleotide, refers to a polynucleotide that differs in nucleotide sequence from a specified wild-type, parental, or reference polynucleotide. The identity of the wild-type, parental, or reference polypeptide or polynucleotide will be apparent from context.

The term “recombinant,” when used in reference to a subject cell, nucleic acid, protein or vector, indicates that the subject has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a riboflavinase is a recombinant vector.

The terms “recovered,” “isolated,” and “separated,” refer to a compound, protein (polypeptides), cell, nucleic acid, amino acid, or other specified material or component that is removed from at least one other material or component with which it is naturally associated as found in nature. An “isolated” polypeptides, thereof, includes, but is not limited to, a culture broth containing secreted polypeptide expressed in a heterologous host cell.

The term “purified” refers to material (e.g., an isolated polypeptide or polynucleotide) that is in a relatively pure state, e.g., at least about 90% pure, at least about 95% pure, at least about 98% pure, or even at least about 99% pure.

The term “enriched” refers to material (e.g., an isolated polypeptide or polynucleotide) that is in about 50% pure, at least about 60% pure, at least about 70% pure, or even at least about 70% pure.

A “pH range,” with reference to an enzyme, refers to the range of pH values under which the enzyme exhibits catalytic activity.

The terms “pH stable” and “pH stability,” with reference to an enzyme, relate to the ability of the enzyme to retain activity over a wide range of pH values for a predetermined period of time (e.g., 15 min., 30 min., 1 hour).

The term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The term “nucleic acid” encompasses DNA, RNA, heteroduplexes, and synthetic molecules capable of encoding a polypeptide. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

“Hybridization” refers to the process by which one strand of nucleic acid forms a duplex with, i.e., base pairs with, a complementary strand, as occurs during blot hybridization techniques and PCR techniques. Stringent hybridization conditions are exemplified by hybridization under the following conditions: 65° C. and 0.1×SSC (where 1×SSC=0.15 M NaCl, 0.015 M Na₃ citrate, pH 7.0). Hybridized, duplex nucleic acids are characterized by a melting temperature (Tₘ), where one half of the hybridized nucleic acids are unpaired with the complementary strand. Mismatched nucleotides within the duplex lower the Tₘ. Very stringent hybridization conditions involve 68° C. and 0.1×SSC

A “synthetic” molecule is produced by in vitro chemical or enzymatic synthesis rather than by an organism.

The terms “transformed,” “stably transformed,” and “transgenic,” used with reference to a cell means that the cell contains a non-native (e.g., heterologous) nucleic acid sequence integrated into its genome or carried as an episome that is maintained through multiple generations.

The term “introduced” in the context of inserting a nucleic acid sequence into a cell, means “transfection”, “transformation” or “transduction,” as known in the art.

A “host strain” or “host cell” is an organism into which an expression vector, phage, virus, or other DNA construct, including a polynucleotide encoding a polypeptide of interest (e.g., an riboflavinase) has been introduced. Exemplary host strains are microorganism cells (e.g., bacteria, filamentous fungi, and yeast) capable of expressing the polypeptide of interest. The term “host cell” includes protoplasts created from cells.

The term “heterologous” with reference to a polynucleotide or protein refers to a polynucleotide or protein that does not naturally occur in a host cell.

The term “endogenous” with reference to a polynucleotide or protein refers to a polynucleotide or protein that occurs naturally in the host cell.

The term “expression” refers to the process by which a polypeptide is produced based on a nucleic acid sequence. The process includes both transcription and translation.

A “selective marker” or “selectable marker” refers to a gene capable of being expressed in a host to facilitate selection of host cells carrying the gene. Examples of selectable markers include but are not limited to antimicrobials (e.g., hygromycin, bleomycin, or chloramphenicol) and/or genes that confer a metabolic advantage, such as a nutritional advantage on the host cell.

A “vector” refers to a polynucleotide sequence designed to introduce nucleic acids into one or more cell types. Vectors include cloning vectors, expression vectors, shuttle vectors, plasmids, phage particles, cassettes and the like.

An “expression vector” refers to a DNA construct comprising a DNA sequence encoding a polypeptide of interest, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

An “His-tag” is a consecutive sequence of several, normally six, histidine amino acids added recombinantly to either C- or N-terminal of the parent enzyme polypeptide sequence, which may enable affinity purification without any expected change in enzyme functionality.

The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequences.

A “signal sequence” is a sequence of amino acids attached to the N-terminal portion of a protein, which facilitates the secretion of the protein outside the cell. The mature form of an extracellular protein lacks the signal sequence, which is cleaved off during the secretion process.

“Biologically active” refers to a sequence having a specified biological activity, such an enzymatic activity.

The term “specific activity” refers to the number of moles of substrate that can be converted to product by an enzyme or enzyme preparation per unit time under specific conditions. Specific activity is generally expressed as units (U)/mg of protein.

As used herein, “percent sequence identity” means that a particular sequence has at least a certain percentage of amino acid residues identical to those in a specified reference sequence, when aligned using the CLUSTAL W algorithm with default parameters. See Thompson et al. (1994) Nucleic Acids Res. 22:4673-4680. Default parameters for the CLUSTAL W algorithm are:

Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF.

Deletions are counted as non-identical residues, compared to a reference sequence. Deletions occurring at either terminus are included. For example, a variant with five amino acid deletions of the C-terminus of the mature 617 residue polypeptide would have a percent sequence identity of 99% (612/617 identical residues×100, rounded to the nearest whole number) relative to the mature polypeptide. Such a variant would be encompassed by a variant having “at least 99% sequence identity” to a mature polypeptide.

“Fused” polypeptide sequences are connected, i.e., operably linked, via a peptide bond between two subject polypeptide sequences.

The term “filamentous fungi” refers to all filamentous forms of the subdivision Eumycotina, particularly Pezizomycotina species.

The term “about” refers to ±5% to the referenced value.

As used herein, the term “malt beverage” includes such foam forming fermented malt beverages as full malted beer, ale, dry beer, near beer, light beer, low alcohol beer, low calorie beer, porter, bock beer, stout, malt liquor, non-alcoholic malt liquor and the like. The term “malt beverages” also includes alternative malt beverages such as fruit flavoured malt beverages, e.g., citrus flavoured, such as lemon-, orange-, lime-, or berry-flavoured malt beverages, liquor flavoured malt beverages, e.g., vodka-, rum-, or tequila-flavoured malt liquor, or coffee flavoured malt beverages, such as caffeine-flavoured malt liquor, and the like.

As used herein, the term “beer” traditionally refers to an alcoholic beverage derived from malt, which is derived from barley, and optionally adjuncts, such as cereal grains, and flavoured with hops. Beer can be made from a variety of grains by essentially the same process. All grain starches are glucose homopolymers in which the glucose residues are linked by either alpha-1,4- or alpha-1,6-bonds, with the former predominating. The process of making fermented malt beverages is commonly referred to as brewing. The principal raw materials used in making these beverages are water, hops and malt. In addition, adjuncts such as common corn grits, refined corn grits, rice, sorghum, refined corn starch, barley, barley starch, dehusked barley, wheat, wheat starch, torrified cereal, cereal flakes, rye, oats, potato, tapioca, and syrups, such as corn syrup, sugar cane syrup, inverted sugar syrup, barley and/or wheat syrups, and the like may be used as a source of starch or fermentable sugar types. The starch will eventually be converted into dextrins and fermentable sugars. For a number of reasons, the malt, which is produced principally from selected varieties of barley, has the greatest effect on the overall character and quality of the beer. First, the malt is the primary flavouring agent in beer. Second, the malt provides the major portion of the fermentable sugar. Third, the malt provides the proteins, which will contribute to the body and foam character of the beer. Fourth, the malt provides the necessary enzymatic activity during mashing.

As used herein, the “process for making beer” is one that is well known in the art, but briefly, it involves five steps: (a) adjunct cooking and/or mashing (b) wort separation and extraction (c) boiling and hopping of wort (d) cooling, fermentation and storage, and (e) maturation, processing and packaging. In the first step, milled or crushed malt is mixed with water and held for a period of time under controlled temperatures to permit the enzymes present in the malt to, for example, convert the starch present in the malt into fermentable sugars. In the second step, the mash is transferred to a “lauter tun” or mash filter where the liquid is separated from the grain residue. This sweet liquid is called “wort” and the left over grain residue is called “spent grain”. The mash is typically subjected to an extraction during mash separation, which involves adding water to the mash in order to recover the residual soluble extract from the spent grain. In the third step, the wort is boiled vigorously. This sterilizes the wort and helps to develop the colour, flavour and odour. Hops are added at some point during the boiling. In the fourth step, the wort is cooled and transferred to a fermenter, which either contains the yeast or to which yeast is added. The yeast converts the sugars by fermentation into alcohol and carbon dioxide gas; at the end of fermentation the fermenter is chilled or the fermenter may be chilled to stop fermentation. The yeast flocculates and is removed. In the last step, the beer is cooled and stored for a period of time, during which the beer clarifies and its flavour develops, and any material that might impair the appearance, flavour and shelf life of the beer settles out. Prior to packaging, the beer is carbonated and, optionally, filtered and pasteurized. After fermentation, a beverage is obtained which usually contains from about 2% to about 10% alcohol by weight. The non-fermentable carbohydrates are not converted during fermentation and form the majority of the dissolved solids in the final beer. This residue remains because of the inability of malt enzymes to hydrolyse the alpha-1,6-linkages of the starch and fully degrade the non-starch polysaccharides. The non-fermentable carbohydrates contribute less than 50 kilocalories per 12 ounces of a lager beer.

As used herein, the “process for making beer” may further be applied in the mashing of any grist.

As used herein, the term “riboflavin-like compounds” is defined as compounds containing an isoalloxazine three ring moiety. Examples include riboflavin, riboflavin-5′-phosphate (also known as flavin mononucleotide; FMN), flavin adenine dinucleotide (FAD). Furthermore, these compounds are also known as flavin nucleotides and function as prosthetic groups of oxidation-reduction enzymes.

As used herein the term “riboflavinase” is defined as an enzyme capable of hydrolyzing, converting or rearranging riboflavin or riboflavin-like compounds in such a way that the photo-sensitizing action of riboflavin and riboflavin-like compounds is modified, lessened, reduced, eliminated and/or inhibited.

As used herein the term “riboflavin hydrolase” is defined as an enzyme that hydrolyzes riboflavin and riboflavin-like compounds, including without limitation lyases (EC 4.3) and nucleosidases (EC 3.2.2). Under some circumstances, the riboflavin hydrolase may produce lumichrone and ribotol as end products.

As used herein the term “riboflavin reductase” is defined as an enzyme the reduces riboflavin and riboflavin-like compounds, including without limitation flavin reductases (EC 1.5.1.30).

As used herein the term “riboflavin destructase” or “flavin destructase” is defined herein as an enzyme the catalyzes the conversion of flavin mononucleotide (FMN) to 5,6-dimethylbenzimidazole (DMB). One example of such an enzyme is the BluB enzymes (SmeBluB1 (SEQ ID NO:2) and PspBluB1 (SEQ ID NO:4)) disclosed herein in accordance with an aspect of the present invention. Under certain conditions, it is possible that the non-phosphorylated counterpart to FNM, being riboflavin, also may be converted by a flavin destructase into DMB.

### Additional Mutations

In some embodiments, the present riboflavinases further include one or more mutations that provide a further performance or stability benefit. Exemplary performance benefits include but are not limited to increased thermal stability, increased storage stability, increased solubility, an altered pH profile, increased specific activity, modified substrate specificity, modified substrate binding, modified pH-dependent activity, modified pH-dependent stability, increased oxidative stability, and increased expression. In some cases, the performance benefit is realized at a relatively low temperature. In some cases, the performance benefit is realized at relatively high temperature.

Furthermore, the present riboflavinases may include any number of conservative amino acid substitutions. Exemplary conservative amino acid substitutions are listed in the following Table.

TABLE 1
Conservative amino acid substitutions

| For Amino Acid | Code | Replace with any of |
|---|---|---|
| Alanine | A | D-Ala, Gly, beta-Ala, |
| | | L-Cys, D-Cys |
| Arginine | R | D-Arg, Lys, D-Lys, |
| | | homo-Arg, D-homo-Arg, |
| | | Met, Ile, D-Met, D-Ile, |
| | | Orn, D-Orn |
| Asparagine | N | D-Asn, Asp, D-Asp, Glu, |
| | | D-Glu, Gln, D-Gln |
| Aspartic Acid | D | D-Asp, D-Asn, Asn, Glu, |
| | | D-Glu, Gln, D-Gln |
| Cysteine | C | D-Cys, S-Me-Cys, Met, |
| | | D-Met, Thr, D-Thr |
| Glutamine | Q | D-Gln, Asn, D-Asn, Glu, |
| | | D-Glu, Asp, D-Asp |
| Glutamic Acid | E | D-Glu, D-Asp, Asp, Asn, |
| | | D-Asn, Gln, D-Gln |
| Glycine | G | Ala, D-Ala, Pro, D-Pro, |
| | | b-Ala, Acp |
| Isoleucine | I | D-Ile, Val, D-Val, Leu, |
| | | D-Leu, Met, D-Met |
| Leucine | L | D-Leu, Val, D-Val, Leu, |
| | | D-Leu, Met, D-Met |
| Lysine | K | D-Lys, Arg, D-Arg, |
| | | homo-Arg, D-homo-Arg, |
| | | Met, D-Met, Ile, D-Ile, |
| | | Orn, D-Orn |
| Methionine | M | D-Met, S-Me-Cys, Ile, |
| | | D-Ile, Leu, D-Leu, Val, |
| | | D-Val |
| Phenylalanine | F | D-Phe, Tyr, D-Thr, |
| | | L-Dopa, His, D-His, Trp, |
| | | D-Trp, Trans-3,4, or 5- |
| | | phenylproline, cis-3,4, |
| | | or 5-phenylproline |
| Proline | P | D-Pro, L-I-thioazolidine- |
| | | 4-carboxylic acid, D-or |
| | | L-1-oxazolidine-4- |
| | | carboxylic acid |
| Serine | S | D-Ser, Thr, D-Thr, |
| | | allo-Thr, Met, D-Met, |
| | | Met(O), D-Met(O), L-Cys, |
| | | D-Cys |
| Threonine | T | D-Thr, Ser, D-Ser, |
| | | allo-Thr, Met, D-Met, |
| | | Met(O), D-Met(O), Val, |
| | | D-Val |
| Tyrosine | Y | D-Tyr, Phe, D-Phe, |
| | | L-Dopa, His, D-His |
| Valine | V | D-Val, Leu, D-Leu, Ile, |
| | | D-Ile, Met, D-Met |

The reader will appreciate that some of the above mentioned conservative mutations can be produced by genetic manipulation, while others are produced by introducing synthetic amino acids into a polypeptide by genetic or other means.

The present riboflavinase may be “precursor,” “immature,” or “full-length,” in which case they include a signal sequence, or “mature,” in which case they lack a signal sequence. Mature forms of the polypeptides are generally the most useful. Unless otherwise noted, the amino acid residue numbering used herein refers to the mature forms of the respective riboflavinase polypeptides. The present riboflavinase polypeptides may also be truncated to remove the N or C-termini, so long as the resulting polypeptides retain riboflavinase activity. In addition, riboflavinase enzymes may be active fragments derived from a longer amino acid sequence. Active fragments are characterized by retaining some or all of the activity of the full length enzyme but have deletions from the N-terminus, from the C-terminus or internally or combinations thereof.

The present riboflavinase may be a “chimeric” or “hybrid” polypeptide, in that it includes at least a portion of a first riboflavinase polypeptide, and at least a portion of a second riboflavinase polypeptide. The present riboflavinase may further include heterologous signal sequence, an epitope to allow tracking or purification, or the like. Exemplary heterologous signal sequences are from B. licheniformis amylase (LAT), B. subtilis (AmyE or AprE), and Streptomyces CelA.

### Production of Variant Riboflavinases

The present riboflavinase can be produced in host cells, for example, by secretion or intracellular expression. A cultured cell material (e.g., a whole-cell broth) comprising a riboflavinase can be obtained following secretion of the riboflavinase into the cell medium. Optionally, the riboflavinase can be isolated from the host cells, or even isolated from the cell broth, depending on the desired purity of the final riboflavinase. A gene encoding a riboflavinase can be cloned and expressed according to methods well known in the art. Suitable host cells include bacterial, fungal (including yeast and filamentous fungi), and plant cells (including algae). Particularly useful host cells include Aspergillus niger, Aspergillus oryzae or Trichoderma reesei. Other host cells include bacterial cells, e.g., Bacillus subtilis or B. licheniformis, as well as Streptomyces, E. Coli.

The host cell further may express a nucleic acid encoding a homologous or heterologous riboflavinase, i.e., a riboflavinase that is not the same species as the host cell, or one or more other enzymes. The riboflavinase may be a variant riboflavinase. Additionally, the host may express one or more accessory enzymes, proteins, peptides.

### Vectors

A DNA construct comprising a nucleic acid encoding a riboflavinase can be constructed to be expressed in a host cell. Because of the well-known degeneracy in the genetic code, variant polynucleotides that encode an identical amino acid sequence can be designed and made with routine skill. It is also well-known in the art to optimize codon use for a particular host cell. Nucleic acids encoding riboflavinase can be incorporated into a vector. Vectors can be transferred to a host cell using well-known transformation techniques, such as those disclosed below.

The vector may be any vector that can be transformed into and replicated within a host cell. For example, a vector comprising a nucleic acid encoding a riboflavinase can be transformed and replicated in a bacterial host cell as a means of propagating and amplifying the vector. The vector also may be transformed into an expression host, so that the encoding nucleic acids can be expressed as a functional riboflavinase. Host cells that serve as expression hosts can include filamentous fungi, for example. The Fungal Genetics Stock Center (FGSC) Catalogue of Strains lists suitable vectors for expression in fungal host cells. See FGSC, Catalogue of Strains, University of Missouri, at www.fgsc.net (last modified Jan. 17, 2007). A representative vector is pJG153, a promoterless Cre expression vector that can be replicated in a bacterial host. See Harrison et al. (June 2011) Applied Environ. Microbiol. 77: 3916-22. pJG153 can be modified with routine skill to comprise and express a nucleic acid encoding a riboflavinase.

A nucleic acid encoding a riboflavinase can be operably linked to a suitable promoter, which allows transcription in the host cell. The promoter may be any DNA sequence that shows transcriptional activity in the host cell of choice and may be derived from genes encoding proteins either homologous or heterologous to the host cell. Exemplary promoters for directing the transcription of the DNA sequence encoding a riboflavinase, especially in a bacterial host, are the promoter of the lac operon of E. coli, the Streptomyces coelicolor agarase gene dagA or celA promoters, the promoters of the Bacillus licheniformis α-amylase gene (amyL), the promoters of the Bacillus stearothermophilus maltogenic amylase gene (amyM), the promoters of the Bacillus amyloliquefaciens α-amylase (amyQ), the promoters of the Bacillus subtilis xylA and xylB genes etc. For transcription in a fungal host, examples of useful promoters are those derived from the gene encoding Aspergillus oryzae TAKA amylase, Rhizomucor miehei aspartic proteinase, Aspergillus niger neutral α-amylase, A. niger acid stable α-amylase, A. niger glucoamylase, Rhizomucor miehei lipase, A. oryzae alkaline protease, A. oryzae triose phosphate isomerase, or A. nidulans acetamidase. When a gene encoding a riboflavinase is expressed in a bacterial species such as E. coli, a suitable promoter can be selected, for example, from a bacteriophage promoter including a T7 promoter and a phage lambda promoter. Examples of suitable promoters for the expression in a yeast species include but are not limited to the Gal 1 and Gal 10 promoters of Saccharomyces cerevisiae and the Pichia pastoris AOX1 or AOX2 promoters. cbh1 is an endogenous, inducible promoter from T. reesei. See Liu et al. (2008) “Improved heterologous gene expression in Trichoderma reesei by cellobiohydrolase I gene (cbh1) promoter optimization,” Acta Biochim. Biophys. Sin (Shanghai) 40(2): 158-65.

The coding sequence can be operably linked to a signal sequence. The DNA encoding the signal sequence may be the DNA sequence naturally associated with the riboflavinase gene to be expressed or from a different Genus or species. A signal sequence and a promoter sequence comprising a DNA construct or vector can be introduced into a fungal host cell and can be derived from the same source. For example, the signal sequence is the cbh1 signal sequence that is operably linked to a cbh1 promoter.

An expression vector may also comprise a suitable transcription terminator and, in eukaryotes, polyadenylation sequences operably linked to the DNA sequence encoding a variant riboflavinase. Termination and polyadenylation sequences may suitably be derived from the same sources as the promoter.

The vector may further comprise a DNA sequence enabling the vector to replicate in the host cell. Examples of such sequences are the origins of replication of plasmids pUC19, pACYC177, pUB110, pE194, pAMB1, and pIJ702.

The vector may also comprise a selectable marker, e.g., a gene the product of which complements a defect in the isolated host cell, such as the dal genes from B. subtilis or B. licheniformis, or a gene that confers antibiotic resistance such as, e.g., ampicillin, kanamycin, chloramphenicol or tetracycline resistance. Furthermore, the vector may comprise Aspergillus selection markers such as amdS, argB, niaD and xxsC, a marker giving rise to hygromycin resistance, or the selection may be accomplished by co-transformation, such as known in the art. See e.g., International PCT Application WO 91/17243.

Intracellular expression may be advantageous in some respects, e.g., when using certain bacteria or fungi as host cells to produce large amounts of riboflavinase for subsequent enrichment or purification. Extracellular secretion of riboflavinase into the culture medium can also be used to make a cultured cell material comprising the isolated riboflavinase.

The expression vector typically includes the components of a cloning vector, such as, for example, an element that permits autonomous replication of the vector in the selected host organism and one or more phenotypically detectable markers for selection purposes. The expression vector normally comprises control nucleotide sequences such as a promoter, operator, ribosome binding site, translation initiation signal and optionally, a repressor gene or one or more activator genes. Additionally, the expression vector may comprise a sequence coding for an amino acid sequence capable of targeting the riboflavinase to a host cell organelle such as a peroxisome, or to a particular host cell compartment. Such a targeting sequence includes but is not limited to the sequence, SKL. For expression under the direction of control sequences, the nucleic acid sequence of the riboflavinase is operably linked to the control sequences in proper manner with respect to expression.

The procedures used to ligate the DNA construct encoding a riboflavinase, the promoter, terminator and other elements, respectively, and to insert them into suitable vectors containing the information necessary for replication, are well known to persons skilled in the art (see, e.g., Sambrook et al., MOLECULAR CLONING: A LABORATORY MANUAL, 2^{nd} ed., Cold Spring Harbor, 1989, and 3rd ed., 2001).

### Transformation and Culture of Host Cells

An isolated cell, either comprising a DNA construct or an expression vector, is advantageously used as a host cell in the recombinant production of a riboflavinase. The cell may be transformed with the DNA construct encoding the enzyme, conveniently by integrating the DNA construct (in one or more copies) in the host chromosome. This integration is generally considered to be an advantage, as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector as described above in connection with the different types of host cells.

Examples of suitable bacterial host organisms are Gram positive bacterial species such as Bacillaceae including Bacillus subtilis, Bacillus licheniformis, Bacillus lentus, Bacillus brevis, Geobacillus (formerly Bacillus) stearothermophilus, Bacillus alkalophilus, Bacillus amyloliquefaciens, Bacillus coagulans, Bacillus lautus, Bacillus megaterium, and Bacillus thuringiensis; Streptomyces species such as Streptomyces murinus; lactic acid bacterial species including Lactococcus sp. such as Lactococcus lactis; Lactobacillus sp. including Lactobacillus reuteri; Leuconostoc sp.; Pediococcus sp.; and Streptococcus sp. Alternatively, strains of a Gram negative bacterial species belonging to Enterobacteriaceae including E. coli, or to Pseudomonadaceae can be selected as the host organism.

A suitable yeast host organism can be selected from the biotechnologically relevant yeasts species such as but not limited to yeast species such as Pichia sp., Hansenula sp., or Kluyveromyces, Yarrowinia, Schizosaccharomyces species or a species of Saccharomyces, including Saccharomyces cerevisiae or a species belonging to Schizosaccharomyces such as, for example, S. pombe species. A strain of the methylotrophic yeast species, Pichia pastoris, can be used as the host organism. Alternatively, the host organism can be a Hansenula species. Suitable host organisms among filamentous fungi include species of Aspergillus, e.g., Aspergillus niger, Aspergillus oryzae, Aspergillus tubigensis, Aspergillus awamori, or Aspergillus nidulans. Alternatively, strains of a Fusarium species, e.g., Fusarium oxysporum or of a Rhizomucor species such as Rhizomucor miehei can be used as the host organism. Other suitable strains include Thermomyces and Mucor species. In addition, Trichoderma sp. can be used as a host. A suitable procedure for transformation of Aspergillus host cells includes, for example, that described in EP 238023. A riboflavinase expressed by a fungal host cell can be glycosylated, i.e., will comprise a glycosyl moiety. The glycosylation pattern can be the same or different as present in the wild-type riboflavinase. The type and/or degree of glycosylation may impart changes in enzymatic and/or biochemical properties.

It is advantageous to delete genes from expression hosts, where the gene deficiency can be cured by the transformed expression vector. Known methods may be used to obtain a fungal host cell having one or more inactivated genes. Gene inactivation may be accomplished by complete or partial deletion, by insertional inactivation or by any other means that renders a gene nonfunctional for its intended purpose, such that the gene is prevented from expression of a functional protein. Any gene from a Trichoderma sp. or other filamentous fungal host that has been cloned can be deleted, for example, cbh1, cbh2, egl1, and egl2 genes. Gene deletion may be accomplished by inserting a form of the desired gene to be inactivated into a plasmid by methods known in the art.

Introduction of a DNA construct or vector into a host cell includes techniques such as transformation; electroporation; nuclear microinjection; transduction; transfection, e.g., lipofection mediated and DEAE-Dextrin mediated transfection; incubation with calcium phosphate DNA precipitate; high velocity bombardment with DNA-coated microprojectiles; and protoplast fusion. General transformation techniques are known in the art. See, e.g., Sambrook et al. (2001), supra. The expression of heterologous protein in Trichoderma is described, for example, in U.S. Pat. No. 6,022,725. Reference is also made to Cao et al. (2000) Science 9:991-1001 for transformation of Aspergillus strains. Genetically stable transformants can be constructed with vector systems whereby the nucleic acid encoding a riboflavinase is stably integrated into a host cell chromosome. Transformants are then selected and purified by known techniques.

The preparation of Trichoderma sp. for transformation, for example, may involve the preparation of protoplasts from fungal mycelia. See Campbell et al. (1989) Curr. Genet. 16: 53-56. The mycelia can be obtained from germinated vegetative spores. The mycelia are treated with an enzyme that digests the cell wall, resulting in protoplasts. The protoplasts are protected by the presence of an osmotic stabilizer in the suspending medium. These stabilizers include sorbitol, mannitol, potassium chloride, magnesium sulfate, and the like. Usually the concentration of these stabilizers varies between 0.8 M and 1.2 M, e.g., a 1.2 M solution of sorbitol can be used in the suspension medium.

Uptake of DNA into the host Trichoderma sp. strain depends upon the calcium ion concentration. Generally, between about 10-50 mM CaCl₂ is used in an uptake solution. Additional suitable compounds include a buffering system, such as TE buffer (10 mM Tris, pH 7.4; 1 mM EDTA) or 10 mM MOPS, pH 6.0 and polyethylene glycol. The polyethylene glycol is believed to fuse the cell membranes, thus permitting the contents of the medium to be delivered into the cytoplasm of the Trichoderma sp. strain. This fusion frequently leaves multiple copies of the plasmid DNA integrated into the host chromosome.

Usually transformation of Trichoderma sp. uses protoplasts or cells that have been subjected to a permeability treatment, typically at a density of 10⁵ to 10⁷/mL, particularly 2×10⁶/mL. A volume of 100 μL of these protoplasts or cells in an appropriate solution (e.g., 1.2 M sorbitol and 50 mM CaCl₂) may be mixed with the desired DNA. Generally, a high concentration of PEG is added to the uptake solution. From 0.1 to 1 volume of 25% PEG 4000 can be added to the protoplast suspension; however, it is useful to add about 0.25 volumes to the protoplast suspension. Additives, such as dimethyl sulfoxide, heparin, spermidine, potassium chloride and the like, may also be added to the uptake solution to facilitate transformation. Similar procedures are available for other fungal host cells. See, e.g., U.S. Pat. No. 6,022,725.

### Expression

A method of producing a riboflavinase may comprise cultivating a host cell as described above under conditions conducive to the production of the enzyme and recovering the enzyme from the cells and/or culture medium.

The medium used to cultivate the cells may be any conventional medium suitable for growing the host cell in question and obtaining expression of a riboflavinase. Suitable media and media components are available from commercial suppliers or may be prepared according to published recipes (e.g., as described in catalogues of the American Type Culture Collection).

An enzyme secreted from the host cells can be used in a whole broth preparation. In the present methods, the preparation of a spent whole fermentation broth of a recombinant microorganism can be achieved using any cultivation method known in the art resulting in the expression of a riboflavinase. Fermentation may, therefore, be understood as comprising shake flask cultivation, small- or large-scale fermentation (including continuous, batch, fed-batch, or solid state fermentations) in laboratory or industrial fermenters performed in a suitable medium and under conditions allowing the riboflavinase to be expressed or isolated. The term “spent whole fermentation broth” is defined herein as unfractionated contents of fermentation material that includes culture medium, extracellular proteins (e.g., enzymes), and cellular biomass. It is understood that the term “spent whole fermentation broth” also encompasses cellular biomass that has been lysed or permeabilized using methods well known in the art.

An enzyme secreted from the host cells may conveniently be recovered from the culture medium by well-known procedures, including separating the cells from the medium by centrifugation or filtration, and precipitating proteinaceous components of the medium by means of a salt such as ammonium sulfate, followed by the use of chromatographic procedures such as ion exchange chromatography, affinity chromatography, or the like. The polynucleotide encoding a riboflavinase in a vector can be operably linked to a control sequence that is capable of providing for the expression of the coding sequence by the host cell, i.e. the vector is an expression vector. The control sequences may be modified, for example by the addition of further transcriptional regulatory elements to make the level of transcription directed by the control sequences more responsive to transcriptional modulators. The control sequences may in particular comprise promoters.

Host cells may be cultured under suitable conditions that allow expression of a riboflavinase. Expression of the enzymes may be constitutive such that they are continually produced, or inducible, requiring a stimulus to initiate expression. In the case of inducible expression, protein production can be initiated when required by, for example, addition of an inducer substance to the culture medium, for example dexamethasone or IPTG or Sophorose. Polypeptides can also be produced recombinantly in an in vitro cell-free system, such as the TNT™ (Promega) rabbit reticulocyte system.

An expression host also can be cultured in the appropriate medium for the host, under aerobic conditions. Shaking or a combination of agitation and aeration can be provided, with production occurring at the appropriate temperature for that host, e.g., from about 25° C. to about 75° C. (e.g., 30° C. to 45° C.), depending on the needs of the host and production of the desired riboflavinase. Culturing can occur from about 12 to about 100 hours or greater (and any hour value there between, e.g., from 24 to 72 hours). Typically, the culture broth is at a pH of about 4.0 to about 8.0, again depending on the culture conditions needed for the host relative to production of a riboflavinase.

### Methods for Enriching and Purifying Riboflavinases

Fermentation, separation, and concentration techniques are well known in the art and conventional methods can be used in order to prepare a riboflavinase polypeptide-containing solution.

After fermentation, a fermentation broth is obtained, the microbial cells and various suspended solids, including residual raw fermentation materials, are removed by conventional separation techniques in order to obtain a riboflavinase solution. Filtration, centrifugation, microfiltration, rotary vacuum drum filtration, ultrafiltration, centrifugation followed by ultra-filtration, extraction, or chromatography, or the like, are generally used.

It is desirable to concentrate a riboflavinase polypeptide-containing solution in order to optimize recovery. Use of unconcentrated solutions requires increased incubation time in order to collect the enriched or purified enzyme precipitate.

The enzyme containing solution is concentrated using conventional concentration techniques until the desired enzyme level is obtained. Concentration of the enzyme containing solution may be achieved by any of the techniques discussed herein. Exemplary methods of enrichment and purification include but are not limited to rotary vacuum filtration and/or ultrafiltration.

The enzyme solution is concentrated into a concentrated enzyme solution until the enzyme activity of the concentrated riboflavinase polypeptide-containing solution is at a desired level.

Concentration may be performed using, e.g., a precipitation agent, such as a metal halide precipitation agent. Metal halide precipitation agents include but are not limited to alkali metal chlorides, alkali metal bromides and blends of two or more of these metal halides. Exemplary metal halides include sodium chloride, potassium chloride, sodium bromide, potassium bromide and blends of two or more of these metal halides. The metal halide precipitation agent, sodium chloride, can also be used as a preservative.

The metal halide precipitation agent is used in an amount effective to precipitate a riboflavinase. The selection of at least an effective amount and an optimum amount of metal halide effective to cause precipitation of the enzyme, as well as the conditions of the precipitation for maximum recovery including incubation time, pH, temperature and concentration of enzyme, will be readily apparent to one of ordinary skill in the art, after routine testing.

Generally, at least about 5% w/v (weight/volume) to about 25% w/v of metal halide is added to the concentrated enzyme solution, and usually at least 8% w/v. Generally, no more than about 25% w/v of metal halide is added to the concentrated enzyme solution and usually no more than about 20% w/v. The optimal concentration of the metal halide precipitation agent will depend, among others, on the nature of the specific riboflavinase polypeptide and on its concentration in the concentrated enzyme solution.

Another alternative way to precipitate the enzyme is to use organic compounds. Exemplary organic compound precipitating agents include: 4-hydroxybenzoic acid, alkali metal salts of 4-hydroxybenzoic acid, alkyl esters of 4-hydroxybenzoic acid, and blends of two or more of these organic compounds. The addition of the organic compound precipitation agents can take place prior to, simultaneously with or subsequent to the addition of the metal halide precipitation agent, and the addition of both precipitation agents, organic compound and metal halide, may be carried out sequentially or simultaneously.

Generally, the organic precipitation agents are selected from the group consisting of alkali metal salts of 4-hydroxybenzoic acid, such as sodium or potassium salts, and linear or branched alkyl esters of 4-hydroxybenzoic acid, wherein the alkyl group contains from 1 to 12 carbon atoms, and blends of two or more of these organic compounds. The organic compound precipitation agents can be, for example, linear or branched alkyl esters of 4-hydroxybenzoic acid, wherein the alkyl group contains from 1 to 10 carbon atoms, and blends of two or more of these organic compounds. Exemplary organic compounds are linear alkyl esters of 4-hydroxybenzoic acid, wherein the alkyl group contains from 1 to 6 carbon atoms, and blends of two or more of these organic compounds. Methyl esters of 4-hydroxybenzoic acid, propyl esters of 4-hydroxybenzoic acid, butyl ester of 4-hydroxybenzoic acid, ethyl ester of 4-hydroxybenzoic acid and blends of two or more of these organic compounds can also be used. Additional organic compounds also include but are not limited to 4-hydroxybenzoic acid methyl ester (named methyl PARABEN), 4-hydroxybenzoic acid propyl ester (named propyl PARABEN), which also are both preservative agents. For further descriptions, see, e.g., U.S. Pat. No. 5,281,526.

Addition of the organic compound precipitation agent provides the advantage of high flexibility of the precipitation conditions with respect to pH, temperature, riboflavinase concentration, precipitation agent concentration, and time of incubation.

The organic compound precipitation agent is used in an amount effective to improve precipitation of the enzyme by means of the metal halide precipitation agent. The selection of at least an effective amount and an optimum amount of organic compound precipitation agent, as well as the conditions of the precipitation for maximum recovery including incubation time, pH, temperature and concentration of enzyme, will be readily apparent to one of ordinary skill in the art, in light of the present disclosure, after routine testing.

Generally, at least about 0.01% w/v of organic compound precipitation agent is added to the concentrated enzyme solution and usually at least about 0.02% w/v. Generally, no more than about 0.3% w/v of organic compound precipitation agent is added to the concentrated enzyme solution and usually no more than about 0.2% w/v.

The concentrated polypeptide solution, containing the metal halide precipitation agent, and the organic compound precipitation agent, can be adjusted to a pH, which will, of necessity, depend on the enzyme to be enriched or purified. Generally, the pH is adjusted at a level near the isoelectric point of the riboflavinase. The pH can be adjusted at a pH in a range from about 2.5 pH units below the isoelectric point (pI) up to about 2.5 pH units above the isoelectric point.

The incubation time necessary to obtain an enriched or purified enzyme precipitate depends on the nature of the specific enzyme, the concentration of enzyme, and the specific precipitation agent(s) and its (their) concentration. Generally, the time effective to precipitate the enzyme is between about 1 to about 30 hours; usually it does not exceed about 25 hours. In the presence of the organic compound precipitation agent, the time of incubation can still be reduced to less about 10 hours and in most cases even about 6 hours.

Generally, the temperature during incubation is between about 4° C. and about 50° C. Usually, the method is carried out at a temperature between about 10° C. and about 45° C. (e.g., between about 20° C. and about 40° C.). The optimal temperature for inducing precipitation varies according to the solution conditions and the enzyme or precipitation agent(s) used.

The overall recovery of enriched or purified enzyme precipitate, and the efficiency with which the process is conducted, is improved by agitating the solution comprising the enzyme, the added metal halide and the added organic compound. The agitation step is done both during addition of the metal halide and the organic compound, and during the subsequent incubation period. Suitable agitation methods include mechanical stirring or shaking, vigorous aeration, or any similar technique.

After the incubation period, the enriched or purified enzyme is then separated from the dissociated pigment and other impurities and collected by conventional separation techniques, such as filtration, centrifugation, microfiltration, rotary vacuum filtration, ultrafiltration, press filtration, cross membrane microfiltration, cross flow membrane microfiltration, or the like. Further enrichment or purification of the enzyme precipitate can be obtained by washing the precipitate with water. For example, the enriched or purified enzyme precipitate is washed with water containing the metal halide precipitation agent, or with water containing the metal halide and the organic compound precipitation agents.

During fermentation, a riboflavinase polypeptide accumulates in the culture broth. For the isolation, enrichment, or purification of the desired riboflavinase, the culture broth is centrifuged or filtered to eliminate cells, and the resulting cell-free liquid is used for enzyme enrichment or purification. In one embodiment, the cell-free broth is subjected to salting out using ammonium sulfate at about 70% saturation; the 70% saturation-precipitation fraction is then dissolved in a buffer and applied to a column such as a Sephadex G-100 column, and eluted to recover the enzyme-active fraction. For further enrichment or purification, a conventional procedure such as ion exchange chromatography may be used.

Enriched or purified enzymes can be made into a final product that is either liquid (solution, slurry) or solid (granular, powder).

In accordance with an aspect of the present invention, it has been discovered that the development of sun-struck off-flavor may be counteracted or inhibited by adding to a food, including a malt beverage at least one riboflavinase enzyme capable of hydrolyzing, converting or rearranging riboflavin or riboflavin-like compounds in such a way that the photo-sensitizing action of riboflavin and riboflavin-like compounds is inhibited. Food includes malt beverages, milk, milk-based dairy product, fermented milk products, ice-cream, vegetable oil, olive oil, soy milk, soy bean oil and oil containing salad dressing. For malt beverages, the riboflavinase enzyme may potentially be added during malting, mashing, fermentation or in the final beer.

In addition, the present riboflavinase may be produced during beer fermentation process by brewers yeast such as Saccharomyces cerevisiae or similar. A suitable brewer's yeast strains having riboflavinase activity or riboflavin destructase activity may be constructed using recombinant DNA cloning vectors or other recombinant techniques. Such, the riboflavinase, riboflavin hydrolase, riboflavin reductase, riboflavin destructase or any combinations hereof would be expressed during beer the fermentation and to reduced, hydrolyse, remove, rearrange or inhibit riboflavin photosensitizing properties.

In accordance with an aspect of the present invention, a method is presented for the inhibition of formation of 3MBT (3-methylbut-ene-thiol) in a food. In preferred aspects of the present invention, an effective amount of a riboflavinase is added to the food. Preferably, the food is a malt beverage. Preferably, the riboflavinase is a riboflavin hydrolase. More preferably, the riboflavin hydrolase is an enzyme having at least 80% sequence identity to MOXRcaE1 (SEQ ID NO:8) or an active fragment thereof or MOXRcaE2 (SEQ ID NO:12) or an active fragment thereof.

In more preferred embodiments, the riboflavin hydrolase is an enzyme having at least 80% sequence identity to MOXRcaE1 or an active fragment thereof. More preferably, the riboflavin hydrolase is an enzyme having at least 90% amino acid sequence identity to MOXRcaE1 or an active fragment thereof. Still more preferably, the riboflavin hydrolase is an enzyme having at least 95% amino acid sequence identity to MOXRcaE1 or an active fragment thereof. In still more preferred embodiments, the riboflavin hydrolase is an enzyme having at least 99% amino acid sequence identity to MOXRcaE1 or an active fragment thereof. In the most preferred embodiments, the riboflavin hydrolase is MOXRcaE1 or an active fragment thereof.

In another aspect of the present invention, the riboflavin hydrolase is an enzyme having at least 80% amino acid sequence identity to MOXRcaE2 or an active fragment thereof. More preferably, the riboflavin hydrolase is an enzyme having at least 90% amino acid sequence identity to MOXRcaE2 or an active fragment thereof. Still more preferably, the riboflavin hydrolase is an enzyme having at least 95% amino acid sequence identity to MOXRcaE2 or an active fragment thereof. Yet more preferably, the riboflavin hydrolase is an enzyme having at least 99% amino acid sequence identity to MOXRcaE2 or an active fragment thereof. In the most preferred embodiments, the riboflavin hydrolase is MOXRcaE2 or an active fragment thereof.

In another aspect of the present invention, in the method of preventing the formation of 3MBT a second riboflavinase is used in addition to the first riboflavinase. The second riboflavinase is preferably a riboflavin reductase. Preferably, the riboflavin reductase is an enzyme having at least 80% amino acid sequence identity to MOXRcaB1 (SEQ ID NO:6) or an active fragment thereof or MOXRcaB2 (SEQ ID NO:10) or an active fragment thereof.

More preferably, the riboflavin reductase is an enzyme having at least 90% amino acid sequence identity to MOXRcaB1 or an active fragment thereof. Yet more preferably, the riboflavin reductase is an enzyme having at least 95% amino acid sequence identity to MOXRcaB1 or an active fragment thereof. Still more preferably, the riboflavin reductase is an enzyme having at least 99% amino acid sequence identity to MOXRcaB1 or an active fragment thereof. In the most preferred embodiments, the riboflavin reductase is MOXRcaB1 or an active fragment thereof.

In another aspect of the invention, the riboflavin reductase is an enzyme having at least 90% amino acid sequence identity to MOXRcaB2 or an active fragment thereof. Yet more preferably, the riboflavin reductase is an enzyme having at least 95% amino acid sequence identity to MOXRcaB2 or an active fragment thereof. Still more preferably, the riboflavin reductase is an enzyme having at least 99% amino acid sequence identity to MOXRcaB2 or an active fragment thereof. In the most preferred embodiments, the riboflavin reductase is MOXRcaB2 or an active fragment thereof.

In accordance with another aspect of the present invention, a malt beverage is presented having an effective amount of a riboflavinase. Preferably, the riboflavinase is a riboflavin hydrolase. More preferably, the riboflavin hydrolase is an enzyme having at least 80% sequence identity to MOXRcaE1 (SEQ ID NO:8) or an active fragment thereof or MOXRcaE2 (SEQ ID NO:12) or an active fragment thereof.

In more preferred embodiments, the riboflavin hydrolase is an enzyme having at least 80% sequence identity to MOXRcaE1 or an active fragment thereof. More preferably, the riboflavin hydrolase is an enzyme having at least 90% amino acid sequence identity to MOXRcaE1 or an active fragment thereof. Still more preferably, the riboflavin hydrolase is an enzyme having at least 95% amino acid sequence identity to MOXRcaE1 or an active fragment thereof. In still more preferred embodiments, the riboflavin hydrolase is an enzyme having at least 99% amino acid sequence identity to MOXRcaE1 or an active fragment thereof. In the most preferred embodiments, the riboflavin hydrolase is MOXRcaE1 or an active fragment thereof.

In another aspect of the present invention, the riboflavin hydrolase is an enzyme having at least 80% amino acid sequence identity to MOXRcaE2 or an active fragment thereof. More preferably, the riboflavin hydrolase is an enzyme having at least 90% amino acid sequence identity to MOXRcaE2 or an active fragment thereof. Still more preferably, the riboflavin hydrolase is an enzyme having at least 95% amino acid sequence identity to MOXRcaE2 or an active fragment thereof. Yet more preferably, the riboflavin hydrolase is an enzyme having at least 99% amino acid sequence identity to MOXRcaE2 or an active fragment thereof. In the most preferred embodiments, the riboflavin hydrolase is MOXRcaE2 or an active fragment thereof.

In another aspect of the present invention, the malt beverage has a second riboflavinase in addition to the first riboflavinase. The second riboflavinase is preferably a riboflavin reductase. Preferably, the riboflavin reductase is an enzyme having at least 80% amino acid sequence identity to MOXRcaB1 (SEQ ID NO:6) or an active fragment thereof or MOXRcaB2 (SEQ ID NO:10) or an active fragment thereof.

More preferably, the riboflavin reductase is an enzyme having at least 90% amino acid sequence identity to MOXRcaB1 or an active fragment thereof. Yet more preferably, the riboflavin reductase is an enzyme having at least 95% amino acid sequence identity to MOXRcaB1 or an active fragment thereof. Still more preferably, the riboflavin reductase is an enzyme having at least 99% amino acid sequence identity to MOXRcaB1 or an active fragment thereof. In the most preferred embodiments, the riboflavin reductase is MOXRcaB1 or an active fragment thereof.

In another aspect of the invention, the riboflavin reductase is an enzyme having at least 90% amino acid sequence identity to MOXRcaB2 or an active fragment thereof. Yet more preferably, the riboflavin reductase is an enzyme having at least 95% amino acid sequence identity to MOXRcaB2 or an active fragment thereof. Still more preferably, the riboflavin reductase is an enzyme having at least 99% amino acid sequence identity to MOXRcaB2 or an active fragment thereof. In the most preferred embodiments, the riboflavin reductase is MOXRcaB2 or an active fragment thereof.

Preferably, the malt beverage of the instant invention is selected from the group consisting of a beer, ale, dry beer, near beer, light beer, low alcohol beer, low calorie beer, porter, bock beer, stout, malt liquor, and non-alcoholic malt liquor. More preferably, the malt beverage is a beer.

In accordance with another aspect of the present invention, the riboflavinase (as used in the method of preventing 3-MBT formation or in a malt beverage as described above) is a riboflavin destructase. Preferably, the riboflavin destructase is an enzyme having at least 80% identity to SmeBluB1 (SEQ ID NO:2) or an active fragment thereof or PspBluB1 (SEQ ID NO:4) or an active fragment thereof.

More preferably, the riboflavin destructase is an enzyme having at least 80% sequence identity to SmeBluB1 or an active fragment thereof. In more preferred embodiments, the riboflavin destructase comprises an enzyme having at least 90% sequence identity to SmeBluB1 or an active fragment thereof. Still more preferably, the riboflavin destructase comprises an enzyme having at least 95% sequence identity to SmeBluB1 or an active fragment thereof. In yet more preferred embodiments the riboflavin destructase is an enzyme having at least 99% sequence identity to SmeBluB1 or an active fragment thereof. In the most preferred embodiments, the riboflavin destructase is SmeBluB1 or an active fragment thereof.

In other preferred embodiments, the riboflavin destructase is an enzyme having at least 80% sequence identity to PspBluB1 or an active fragment thereof. In more preferred embodiments, the riboflavin destructase comprises an enzyme having at least 90% sequence identity to PspBluB1 or an active fragment thereof. Still more preferably, the riboflavin destructase comprises an enzyme having at least 95% sequence identity to PspBluB1 or an active fragment thereof. In yet more preferred embodiments the riboflavin destructase is an enzyme having at least 99% sequence identity to PspBluB1 or an active fragment thereof. In the most preferred embodiments, the riboflavin destructase is PspBluB1 or an active fragment thereof.

The present disclosure is described in further detail in the following examples, which are not in any way intended to limit the scope of the disclosure as claimed. The attached figures are meant to be considered as integral parts of the specification and description of the disclosure. The following examples are offered to illustrate, but not to limit the claimed disclosure.

### Example 1

### Identification and Cloning of SmeBluB1, PspBluB2, MoxRcaB1, MoxRcaE1, MoxRcaB2 and MoxRcaE2

As described in the literature (Taga et al., Nature, 446: 449-453, 2007), a protein from Sinorhizobium meliloti 1021 was demonstrated to conduct the oxygen-dependent transformation of flavin mononucleotide (FMN) to 5,6-dimethylbenzimidazole (DMB) and D-erythrose 4-phosphate (E4P). Its full-length gene nucleotide acid sequence (herein named SmeBluB1), as identified in the NCBI database (NCBI Reference Sequence: NC_003047.1 from 1998826-1999509, complementary), is provided in SEQ ID NO:1. The corresponding protein encoded by the SmeBluB1 gene is shown in SEQ ID NO:2 (NCBI reference sequence: WP 010969508.1).

With SmeBluB1 (SEQ ID NO:2) as the query, a homolog (herein named PspBluB2) that shares 46% protein sequence identity to SmeBluB1, was identified in Paenibacillus sp. Soil724D2. The full-length gene nucleotide acid sequence of PspBluB2, as identified in the NCBI database (NCBI Reference Sequence: NZ_LMRY01000006.1 from 44623 to 45273), is provided in SEQ ID NO:3. The corresponding protein encoded by the PspBluB2 gene is shown in SEQ ID NO:4 (NCBI reference sequence: WP_060645852.1).

As described in the literature (Xu et al., The Journal of Biological Chemistry, 291: 23506-23515, 2016), a gene cluster that is involved in the riboflavin catabolism is identified from Microbacterium maritypicum G10. Within the gene cluster, the Microbacterium maritypicum RcaB is designated as the flavin-reductase. Based on its N-terminal peptide sequence (TTVVT) and C-terminal peptide sequence (APESA) that are derived from the PCR primers described in Xu et al.'s paper (5′-AAAACATATGACGACTGTCGTGACCGA-3′ and 5′-AAAACTCGAGTTACGCGCTCTCGGGAGC-3′, respectively), a homolog (herein named MoxBluB1) was identified from Microbacterium oxydans strain NS234. The nucleotide acid sequence for the full-length MoxRcaB1 gene, as identified in the NCBI database (NCBI Reference Sequence: NZ_LDRQ01000062.1 from 15504 to 16013), is provided in SEQ ID NO:5. The corresponding protein encoded by the MoxRcaB1 gene is shown in SEQ ID NO:6 (GenBank reference sequence: KTR74700).

Within the gene cluster, Microbacterium maritypicum RcaE is designated as the Riboflavin hydrolase. Based on its N-terminal peptide sequence (TDQNT) and C-terminal peptide sequence (TMSRV) that are derived from the PCR primers described in Xu et al.'s paper (5′-AAAACATATGACCGATCAGAACACCGT-3′ and 5′-AAAAGAATTCAGACACGCGACATCGTC-3′, respectively), a homolog (herein named MoxRcaE1) was identified from Microbacterium oxydans strain NS234. The nucleotide acid sequence for the full-length MoxRcaE1 gene, as identified in the NCBI database (NCBI Reference Sequence: NZ_LDRQ01000062.1 from 12536 to 13915), is provided in SEQ ID NO:7. The corresponding protein encoded by the MoxRcaE1 gene is shown in SEQ ID NO:8 (GenBank reference sequence: KTR74697).

With MoxRcaB1 (SEQ ID NO:6) as the query, a homolog (herein named MoxRcaB2) that shares 98% protein sequence identity to MoxRcaB1, was identified from Microbacterium oxydans strain BEL163 RN51. The full-length gene nucleotide acid sequence MoxRcaB2, as identified in the NCBI database (NCBI Reference Sequence: NZ_JYIV01000028.1 from 344286 to 344795), is provided in SEQ ID NO:9. The corresponding protein encoded by the MoxRcaB2 gene is shown in SEQ ID NO:10 (GenBank reference sequence: KJL20664).

With MoxRcaE1 (SEQ ID NO:8) as the query, a homolog (herein named MoxRcaE2) that shares 97% protein sequence identity to MoxRcaE1, was identified from Microbacterium oxydans strain BEL163 RN51. The full-length gene nucleotide acid sequence MoxRcaE2, as identified in the NCBI database (NCBI Reference Sequence: NZ_JYIV01000028.1 from 341313 to 342697), is provided in SEQ ID NO:11. The corresponding protein encoded by the MoxRcaE2 gene is shown in SEQ ID NO:12 (GenBank reference sequence: KJL20661).

### Example 2

### Expression and Purification of SmeBluB1, PspBluB2, MoxRcaB1, MoxRcaE1, MoxRcaB2 and MoxRcaE2

The DNA sequence encoding the full-length PspBluB2 (SEQ ID NO:4), MoxRcaE1 (SEQ ID NO:8) or MoxRcaE2 (SEQ ID NO:12) was synthesized and inserted into Bacillus subtilis expression vector p2JM103BBI (Vogtentanz, Protein Expr Purif, 55:40-52, 2007) by Generay (Shanghai, China). The resulting plasmids were designated p3JM-PspBluB2, p3JM-MoxRcaE1 and p3JM-MoxRcaE2.

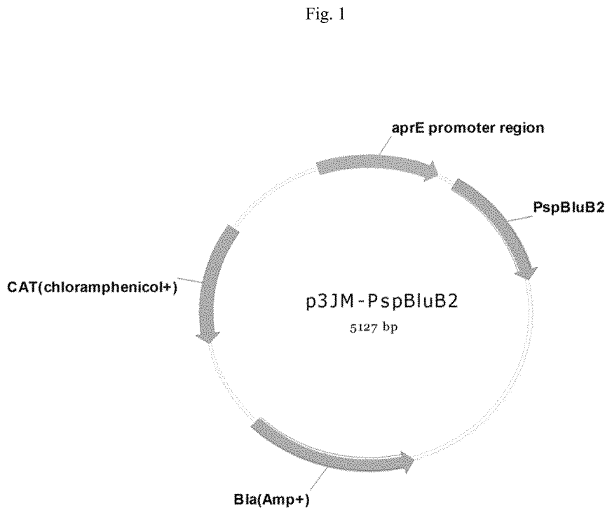

The plasmid map of p3JM-PspBluB2 is provided in FIG. 1; and p3JM-MoxRcaE1 and p3JM-MoxRcaE2 have similar composition with the exception of the inserted gene encoding each gene of interest (GOI). The nucleotide sequences of synthetic PspBluB2, MoxRcaE1 and MoxRcaE2 genes are set forth as SEQ ID NO:13, 14 and 15, respectively. The expression plasmids were then transformed into suitable B. subtilis cells and the transformed cells were cultured on Luria Agar plates supplemented with 5 ppm Chloramphenicol. The correct colony confirmed by PCR was picked and used to inoculated liquid cultures. The fermentation was carried out in 250 mL shake flasks using a MOPS-based defined medium.

The DNA sequence encoding the full-length SmeBluB1 (SEQ ID NO:2), MoxRcaB1 (SEQ ID NO:6) or MoxRcaB2 (SEQ ID NO:10) was synthesized and inserted into E. coli expression vector pET-28b(+) (69865, MilliporeSigma) at NdeI/XhoI site by Generay (Shanghai, China). The resulting plasmids were designated pET-28b-SmeBluB1, pET-28b-MoxRcaB1 and pET-28b-MoxRcaB2.

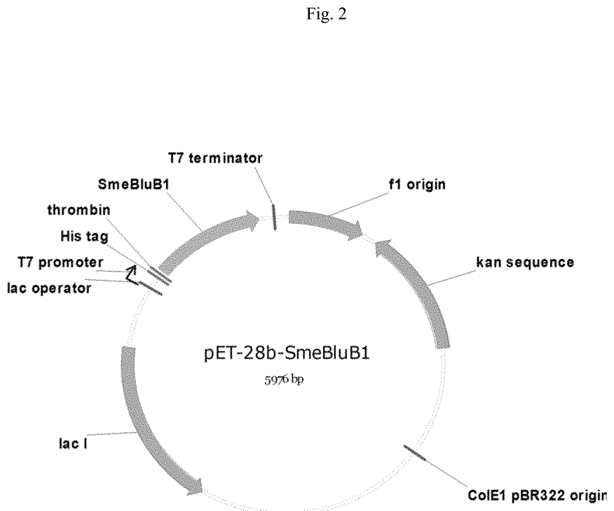

The plasmid map of pET-28b-SmeBluB1 is provided in FIG. 2; and pET-28b-MoxRcaB1 and pET-28b-MoxRcaB2 have similar composition with the exception of the inserted gene encoding each GOI. The nucleotide sequences of synthetic SmeBluB1, MoxRcaB1 or MoxRcaB2 genes are set forth as SEQ ID NO:16, 17 and 18, respectively. And the amino acid sequences expressed from pET-28b-SmeBluB1, pET-28b-MoxRcaB1 and pET-28b-MoxRcaB2, are set forth as SEQ ID NO:19, 20 and 21, respectively. As shown in FIG. 2, the complete expression cassette of pET-28b-SmeBluB1 contains the synthetic nucleotide sequence encoding the SmeBluB1 (SEQ ID NO:2), the N-terminal 6×His-tag followed by the thrombin cleavage peptide. The plasmids were transformed into Rosetta™ 2(DE3)pLysS (71403, MilliporeSigma) and the transformed cells were cultured on Luria Agar plates supplemented with 50 ppm Kanamycin. The correct colony confirmed by PCR was picked and used to inoculated liquid cultures. The fermentation was carried out in 250 mL shake flasks using the MagicMedia™ E. coli Expression Medium (K6803, ThermoFisher).

To purify PspBluB2, the crude from shake flask was concentrated and added ammonium sulfate to the final concentration of 1 M. The solution was loaded onto a HiPrep™ Phenyl FF 16/10 column pre-equilibrated with 20 mM NaPi (pH7.0) supplemented with additional 1 M ammonium sulfate. The target protein was eluted from the column with 0.5 M ammonium sulfate. The corresponding fractions were pooled, concentrated and buffer exchanged into 20 mM NaPi (pH7.0) (Buffer A), using a VivaFlow 200 ultra-filtration device (Sartorius Stedim). The resulting solution was applied to a HiPrep™ Q FF 16/10 column pre-equilibrated with Buffer A. The target protein was eluted from the column with 0.3 M NaCl in buffer A. The fractions containing target protein were pooled, concentrated and subsequently loaded onto a HiLoad™ 26/60 Superdex™ 75 column pre-equilibrated with 20 mM NaPi (pH7.0) supplemented with additional 0.15 M NaCl. The fractions containing target protein were then pooled and concentrated via the 10K Amicon Ultra devices, and stored in 40% glycerol at −20° C. until usage.

To purify MoxRcaE1, the crude from shake flask was concentrated and added ammonium sulfate to the final concentration of 1 M. The solution was loaded onto a HiPrep™ Phenyl FF 16/10 column pre-equilibrated with 20 mM NaPi (pH7.0) supplemented with additional 1 M ammonium sulfate. The target protein was eluted from the column with 0 M ammonium sulfate. The corresponding fractions were pooled, concentrated and buffer exchanged into 20 mM NaPi (pH7.0) (Buffer A), using a VivaFlow 200 ultra-filtration device (Sartorius Stedim). The resulting solution was applied to a HiPrep™ Q FF 16/10 column pre-equilibrated with Buffer A. The target protein was eluted from the column with 0.3 M NaCl in buffer A. The fractions containing target protein were then pooled and concentrated via the 10K Amicon Ultra devices, and stored in 40% glycerol at −20° C. until usage.

To purify MoxRcaE2, the crude from shake flask was concentrated and added ammonium sulfate to the final concentration of 1 M. The solution was loaded onto a HiPrep′ Phenyl FF 16/10 column pre-equilibrated with 20 mM NaPi (pH7.0) supplemented with additional 1 M ammonium sulfate. The target protein was eluted from the column with 0.5 M ammonium sulfate. The corresponding fractions were pooled, concentrated and buffer exchanged into 20 mM NaPi (pH7.0) (Buffer A), using a VivaFlow 200 ultra-filtration device (Sartorius Stedim). The resulting solution was applied to a HiPrep™ Q FF 16/10 column pre-equilibrated with Buffer A. The target protein was eluted from the column with 0.4 M NaCl in buffer A. The fractions containing target protein were then pooled and concentrated via the 10K Amicon Ultra devices, and stored in 40% glycerol at −20° C. until usage.

To purify SmeBluB1, MoxRcaB1 and MoxRcaB2, the cells were harvested by centrifugation and the pellet was re-suspended in lysis buffer (20 mM NaPi pH 7.0, 150 mM NaCl, 0.01% tween-20) and lysed on ice via ultra-sonicator for 20 min (35% power, 20 min, 2 s on/2 s off) (SCIENT2-II D, Ningbo Scientz Biotechnology Co., LTD). The lysate was cleared by centrifugation at 13000 rpm for 30 min (BECKMAN COULTER, Avanti@ J-E). The clarified lysate was applied onto His Trap™ HP 5 mL (GE Healthcare) pre-equilibrated with 20 mM NaPi pH 7.0, 150 mM NaCl. The target protein was eluted from the column with a linear gradient from 0 to 250 mM imidazole in equilibration buffer. The fractions contained target protein was pooled, concentrated and exchanged buffer to equilibration buffer via the 10K Amicon Ultra devices, and stored in 40% glycerol at −20° C. until usage.

### Example 3—Protein Determination Methods

### Protein Determination by Stain Free Imager Criterion

Protein was quantified by SDS-PAGE gel and densitometry using Gel Doc™ EZ imaging system. Reagents used in the assay: Concentrated (2×) Laemmli Sample Buffer (Bio-Rad, Catalogue #161-0737); 26-well XT 4-12% Bis-Tris Gel (Bio-Rad, Catalogue #345-0125); protein markers “Precision Plus Protein Standards” (Bio-Rad, Catalogue #161-0363); protein standard BSA (Thermo Scientific, Catalogue #23208) and SimplyBlue Safestain (Invitrogen, Catalogue # LC 6060. The assay was carried out as follow: In a 96 well-PCR plate 504, diluted enzyme sample were mixed with 50 μL sample buffer containing 2.7 mg DTT. The plate was sealed by Microseal ‘B’ Film from Bio-Rad and was placed into PCR machine to be heated to 70° C. for 10 minutes. After that the chamber was filled by running buffer, gel cassette was set. Then 10 μL of each sample and standard (0.125-1.00 mg/mL BSA) was loaded on the gel and 5 μL of the markers were loaded. After that the electrophoresis was run at 200 V for 45 min. Following electrophoresis the gel was rinsed 3 times for 5 minutes in water, then stained in Safestain overnight and finally destained in water. Then the gel was transferred to Imager. Image Lab software was used for calculation of intensity of each band. By knowing the protein amount of the standard sample, the calibration curve can be made. The amount of sample can be determined by the band intensity and calibration curve. The protein quantification method was employed to prepare samples of riboflavinase enzyme used for assays shown in subsequent examples.

### Example 4—Enzymatic Degradation of Riboflavin in Buffer

The current example serves to demonstrate the enzymatic hydrolysis of riboflavin in a buffered solution. All enzymatic reactions were carried out in potassium phosphate buffer at pH 7.5 and substrate and products were monitored by HPLC. For HPLC analysis, an Agilent 1260 HPLC equipped with a quaternary pump, autosampler, column heater, and diode array detector was used. The system was equipped with a Zorbax XDB-C18 column, temperature was 23° C., flow 1 mL/min, absorbance was monitored at 340 nm and the following gradient elution was used: 100% A (0-2 min), 70% B (2-12 min), 100% A (18-20 min); mobile phase A: H₂O (1 mM ammonium acetate); mobile phase B: MeOH (1 mM ammonium acetate). Data were viewed and processed with ChemStation software DataAnalysis version 4.0 SP 2

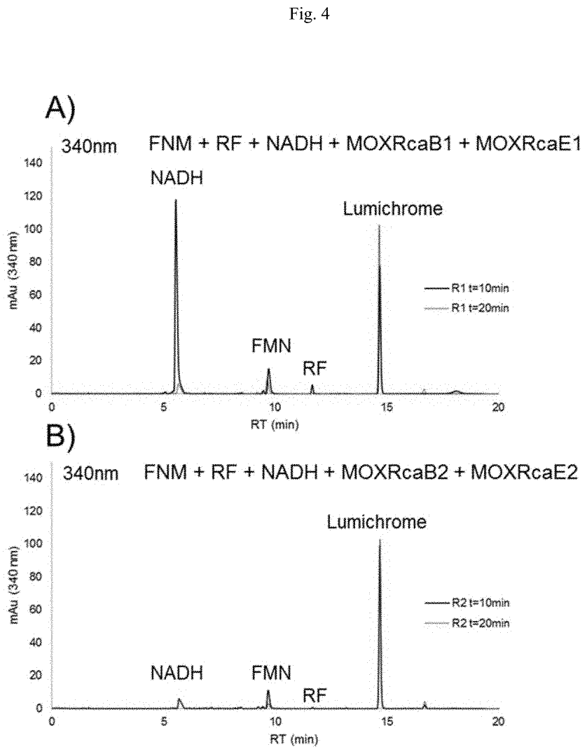

For in vitro assay using the purified MOXRcaE and MOXRcaB enzymes (SEQ ID NO:6, SEQ ID NO:8, SEQ ID NO:10 and SEQ ID NO:12), the following chemical were used: Riboflavin (RF, Sigma-Aldrich, 70% purity, Mw: 376.37, 37.6 mg/mL), Flavin mononucleotide (FMN, Lot # CDS020791, Sigma-Aldrich, MW: 456.34), Lumichrome (Lot # BGBC5866V, Sigma-Aldrich, MW: 242.23) and β-Nicotinamide adenine dinucleotide (NADH, Lot #12165227, Sigma-Aldrich, Mw: 709.40). For in vitro studies MOXRca enzymes (1 μM MOXRcaB and 10 μM MOXRcaE respectively, protein concentration determined as stated in example 3) in different combinations were incubate a reaction mixture of 200 μM FMN, 500 μM RF, 5 mM NADH, in 20 mM sodium phosphate buffer (pH 7.5) at 37 C for 20 min. Control experiments were performed under the same conditions but in the absence of substrate, MOXRcaE, MOXRcaB and NADH, respectively. After 20 min incubation, the reaction was stopped by ultra-filtration (10 kDa cut-off). The filtrate was analyzed by reverse phase HPLC (340 nm) and the relative reduction in riboflavin was quantified. An example of the chromatograms is shown in FIG. 4, where it's also clear that majority of the end products is lumichrome.

The results are shown in table 1. An effective degradation of 76% was obtained under the given conditions described above for both RcaE enzymes (riboflavin hydrolases) in presence of FNM and NADH. No riboflavin degradation was observed for the RcaB enzymes (riboflavin reductase), however in combination with RcaE both combinations (RcaB+RcaE) tested improved degradation to 83 and 84%, respectively.

TABLE 2
In vitro, enzymatic RF degradation in sodium phosphate buffer. The relative
concentration of riboflavin after 20 minutes reaction under assay conditions was quantified
by HPLC (340 nm) and was compared to the blank of just riboflavin (RF).

| | Relative RF concentration |
|---|---|
| Reaction constituents: | in % after 20 min |
| RF | 100 |
| RF + FMN + NADH | 100 |
| RF + FMN + NADH + MOXRcaB1 | 100 |
| RF + FMN + NADH + MOXRcaB2 | 100 |
| RF + FMN + NADH + MOXRcaE1 | 24 |
| RF + FMN + NADH + MOXRcaE2 | 24 |
| RF + FMN + NADH + MOXRcaB1 + MOXRcaE1 | 17 |
| RF + FMN + NADH + MOXRcaB2 + MOXRcaE2 | 16 |

### Example 5—Enzymatic Degradation of Riboflavin in Beer

De-gassed regular German pilsner style beer (5.0% v/v alc., 7 EBC) was prepared protected from sun-light by 2 hours magnetic agitation at RT. A stock solution of 20 mM β-NADH (β-Nicotinamide adenine dinucleotide, Ref 03277372, Roche, Germany) was prepared in a 20 mM Na-phosphate buffer (Merck, Germany) pH 7.0. Enzyme reactions in the de-gassed beer (pH 4.2) were carried out in light-protected 96-well MTP plates (BD Falcon microtest, 96 well, assay plate black) with a total volume of 250 μL sealed with light-protective tape. The purified Rca enzymes (SEQ ID NO:6, SEQ ID NO:8, SEQ ID NO:10 and SEQ ID NO:12) were dosed in different concentrations, in different combinations and with or without NADH in the de-gassed beer and left for 24 hours at 5° C. All samples were filtered in 0.2 μm PVDF filter plates (Corning, N.Y., PVDF MTP) prior to HPLC analysis.

For HPLC analysis, an Agilent 1260 HPLC equipped with a quaternary pump, autosampler, column heater, and fluorescence detector was used. The system was equipped with a Zorbax Eclipse Plus C18 RRHD, particle size 1.8 μm; D×L: 2.1×50 mm, column temperature was 40° C., flow 0.5 mL/min, injection volume: 10 μl and Fluorescence detection with excitation at 274 nm and emission read at 520 nm. Mobile phase A: milli Q water and mobile phase B: methanol was used with the following gradient min/% B: 0/10; 2/20; 5/21.8; 8/50, 9/50; 10/10 and 12/10.

The results are shown in table 2 and it clear that the combinations of RcaB1:RcaE1 and RcaE2:RcaB2 enabled riboflavin (RF) degradation in the beer, with a quantified residual RF content of 89% and 96%, respectively. Complete RF degradation was observed for RcaB1:RcaE1 and RcaE2:RcaB2 in presence of 1000 μM NADH, whereas no degradation was observed for the individual enzymes and NADH.

TABLE 3
Enzymatic RF degradation in regular German pilsner
style beer, pH 4.2. The relative concentration
of riboflavin after 24 hours at 5° C. reaction was
quantified by HPLC and compared to the untreated beer.

| | % Relative |
|---|---|
| | concentration of |
| Samples | riboflavin |
| Beer, | 100 |
| Beer; 1000 μM NADH | 100 |
| Beer; 2 μM MoxRcaB2 | 100 |
| Beer; 20 μM MoxRcaE2 | 100 |
| Beer; 2 μM MoxRcaB1 | 100 |
| Beer; 20 μM MoxRcaE1 | 100 |
| Beer; 2 μtM MoxRcaB2; 20 μM MoxRcaE2 | 96 |
| Beer; 2 μM MoxRcaB2; 20 μM MoxRcaE2; | 0 |
| 1000 μM NADH | |
| Beer; 2 μM MoxRcaB1; 20 μM MoxRcaE1 | 89 |
| Beer; 2 μM MoxRcaB1; 20 μM MoxRcaE1; | 0 |
| 1000 μM NADH | |

### Example 6—Quantification of 3-MBT

The following example describes the method for quantifying 3-MBT in beer. Chemicals: 3-Methylbut-2-ene-1-thiol (3-MBT) was purchased as a 1% solution in triacetin from Chemos GmbH & Co, Regenstauf, Germany (Cat. no. 143379). o-Cresol was purchased from Sigma-Aldrich (Cat. no. C85700). Acetone was purchased from Fisher Scientific (Cat. no. 176800026). Sodium chloride was purchased from Fisher Scientific (Cat. no. S/3120/60).

A 3-MBT stock solution was prepared by diluting the 1% 3-MBT in triacetin 20× in acetone (0.5 mg/mL). A standard solution is prepared by further dilution in acetone 2500× (0.2 μg/ml). An o-cresol stock solution (used as internal standard) was prepared by diluting 40 mg of o-cresol in 200 mL of demineralized water (200 μg/mL). This stock solution was further diluted 40× (5 μg/mL).

From the 3-MBT stock solution, calibration standards were prepared in the range 0-0.3 ng/mL by adding 0 to 10 μL of the standard solution in 6 mL of the sample to be analyzed to which also was added 3.0 g NaCl and 204, of the internal standard solution (16 ng/mL). The calibration standards were prepared in 22 mL headspace vials.

Sample preparation: 6 mL of sample was added to a 22.0 mL headspace vial together with 3 g NaCl and 10 μL of the internal standard stock solution. Analyses were performed in duplicate.

Instrumentation: For this work, a 6890N gas chromatograph coupled to a 5975C mass spectrometer (both from Agilent Technologies) was used. Injection was done using a PAL System from CTC Analytics in the SPME mode. The column installed was a CP-Sil8 CB 30 meter, 320 μm in diameter and coated with a 1.0 μm film (Cat. no. CP7596). The vials were equilibrated at 80° C. for 5 minutes and the gas phase volatiles were adsorbed onto an 85 μm CAR/PDMS SPME-fibre for 20 minutes (Cat. no. 57335-U from Supelco). Subsequently, the volatiles were desorbed at 300° C. for 30 seconds in splitless mode (split flow 5:1 after 6 seconds), and GC/MS data were acquired in the SIM mode (102/68 amu for 3-MBT, 97/68 for 3-Methylthiophene and 107/108 for o-cresol). The oven program was 35° C. for 1 minute, then 10° C./minute to 240° C. for 3 minutes. 3-Methylthiophene is an oxidation product from 3-MBT. The analytical result was reported as the sum of 3-MBT and 3-methylthiophene.

### Example 7—Evaluation of 3-MBT in Beer

The following example describes the evaluation of light induced 3-MBT generation in beer.

Material: A regular German pilsner style beer (5.0% v/v alc., 7 EBC) were kept in a box covered with a towel in the refrigerator until they were used for the experiments (both canned pilsner beer and bottled pilsner beer).

Procedure: Beer from a can of regular German pilsner style beer was gently poured into 3×50 mL Blue Cap flasks labelled 1 to 3, see table 2. The flasks were immediately closed with the mating lids. Beer from a bottle was gently poured into 2×50 mL Blue Cap flasks labelled 4 to 5. The flasks were immediately closed with the mating lids. The strip light (T5 Strip light, 24 W, 6400 K, 1200 lumen, 58 cm length, Nelson Garden) was placed at the table, laying down to lighten the samples from the side of the flasks. Sample 1 and 4 were immediately wrapped in tin foil and put into the refrigerator in a box covered with a towel (0 hour's light exposure). Sample numbers: 3 and 5 were placed from the light source on a mark with a distance to the light source of 12 cm and the light was switched on (for 5 hours' light exposure). After 2 hours of light treatment sample 2 was placed in front of the light strip on marks with a distance to the light source of 12 cm (for 3 hours' light exposure). 5 hours after light treatment was started the light was switched off and all samples were immediately wrapped in tin foil and analyzed for 3-MBT content according to the method described in example 5. This procedure was replicated in two experiments; exp1 and exp2.

TABLE 4
Design of experiment

| | Light exposure (hours) | |
|---|---|---|
| | Regular pilsner | Regular pilsner |
| Sample | beer from Can | beer from Bottle |
| 1 | 0 | |
| 2 | 3 | |
| 3 | 5 | |
| 4 | | 0 |
| 5 | | 5 |

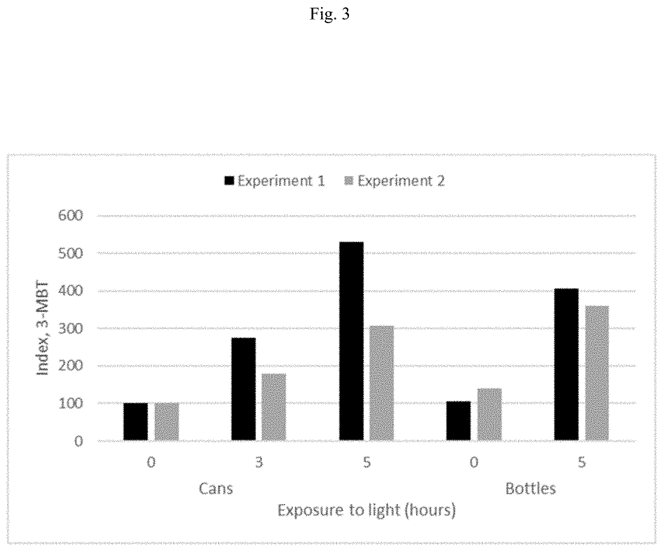

The results from 3-MBT analysis were indexed towards the beer sample taken from the canned beer that was exposed to light for zero hours, for exp1 and exp2, respectively. The results for those two samples were set to Index 100, see FIG. 3.

It was observed that the content of 3-MBT increased with increasing time of exposure to light. The initial content in bottled beer samples were little higher than the observed concentration in the canned beer samples suggesting that beer in the light green bottles were less protected towards light than the canned beer (below are both indexed to 100 on time=0 hours).

### Example 8—Evaluation of 3-MBT in Beer Using Riboflavin Binding Protein

Material: A regular German pilsner style beer (5.0% v/v alc.) was kept in a box covered with a towel in the refrigerator until it was used for the experiment.

Procedure: Beer from a can of regular German pilsner style beer was gently poured into 4×50 mL Blue Cap flasks labelled 1 to 4. The two flasks labelled 1 and 2 were immediately closed with mating lids and wrapped into tin foil. 20 mg of riboflavin binding protein (RfBP, apo-form, Sigma Aldrich, R8628) was added to each of the two flasks labelled 3 and 4 and were immediately closed with mating lids and wrapped into tin foil. All four flasks were put into a refrigerator (5° C.) and kept dark and cold overnight (16 hrs.). The next day the strip light (T5 Strip Light, 24 W, 6400 K, 1200 lumen, 58 cm length, Nelson Garden) was placed at the table, laying down to lighten the samples from the side of the flasks.

Samples labelled 1 and 3 were kept in the refrigerator (0 hour's light exposure). Samples labelled 2 and 4 were placed from the light source on marks with a distance to the light source of 12 cm and the light was switched on (for 4 hours' light exposure). Four hours after the light treatment was started the light was switched off and the samples were immediately wrapped in tin foil and analyzed for 3-MBT content according to the method described in example 5.

Samples labelled 3 and 4 were prepared for centrifugation, 2×400 μl of each sample were added into small tubes with a filter (VIVASPIN 500, membrane 10,000 MWCO PES, Sartorius) and centrifuged for 30 min at 10,000 rpm prior to analysis to remove protein precipitate.

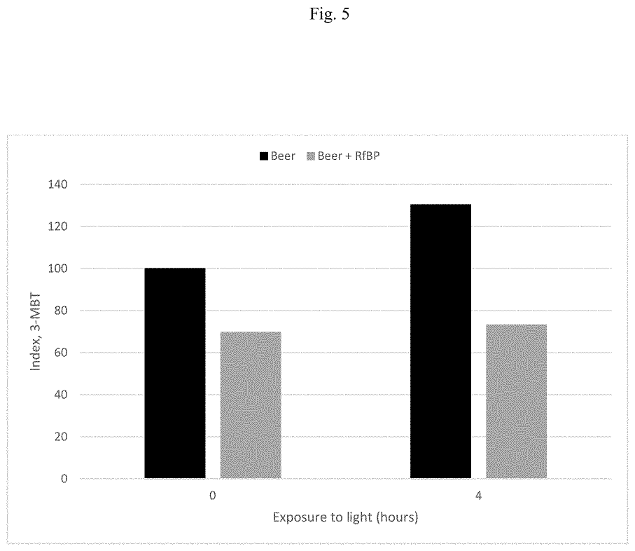

The results from 3-MBT analysis were indexed towards the untreated beer sample that was exposed to light for zero hours. The result for this samples was set to Index 100, FIG. 5.

It was observed that the content of 3-MBT increased with increasing time of exposure to light when the beer was untreated. For beer samples treated with riboflavin binding protein the content of 3-MBT in general was lower and it stayed at a constant level with increasing time of exposure to light suggesting that bound riboflavin could not be part of the 3-MBT formation. Similar or better effect on 3-MBT signal is speculated from beer with completely hydrolyzed riboflavin.

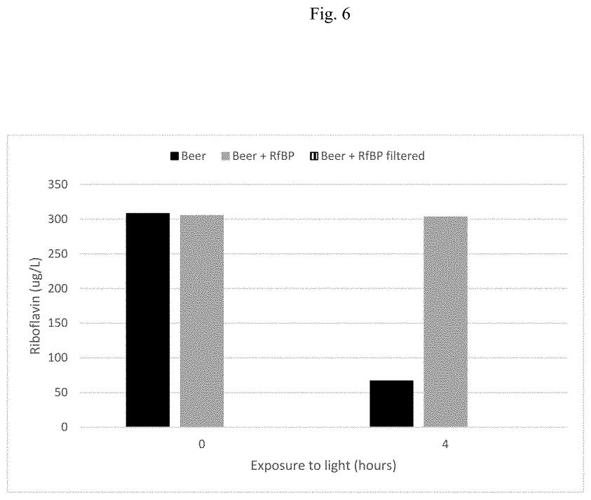

The results from riboflavin analysis is shown in FIG. 6. It was observed that riboflavin content decreased with increasing time of light exposure showing that riboflavin was unstable when induced to light. In beer samples where the riboflavin was bound to the riboflavin binding protein, the riboflavin content stayed at initial level during light exposure of 4 hours suggesting that riboflavin was not degraded by light when bound to the protein. That no riboflavin was present in the filtered beer samples treated with RfBP showed that all riboflavin in the beer samples were bound to the riboflavin binding protein which supported that there was no “free” riboflavin present in the samples which could be photolyzed during the light treatment.

### Example 9—Evaluation of 3-MBT in Beer Using Riboflavin Degrading Enzymes

Material: A regular German pilsner style beer (5.0% v/v alc.) was kept in a box covered with foil in the refrigerator until it was used for the experiment. The enzymes MOXRcaE and MOXRcaB was prepared as described above (SEQ ID NO:6, SEQ ID NO:8, SEQ ID NO:10 and SEQ ID NO:12).

Procedure: Beer from a can of regular German hopped pilsner style beer was degassed for 15 minutes and pH adjusted to pH 6.0 and added to 4.8 mL. β-NADH (β-Nicotinamide adenine dinucleotide, Ref. 03277372, Roche, Germany) was added to all samples in a concentration of 12 μM and separate samples was created with MOXRcaE and MOXRcaB in equimolar (1:1) concentrations with a total enzyme concentration of 2, 12 and 24 μM. Blank or control samples were created by exchanging enzyme addition by ddH2O. the samples (4.8 mL) was poured in and into 4.8 mL Wheaton flint glass vials and left 24 hrs at 14° C. to complete riboflavin degradation. A small aliquot was taken from the sample for HPLC riboflavin quantification as described in example 5 and the remaining was illuminated and following analyzed for 3-MBT development. All vials were put into an aluminum coated box with the dimension of 220×330×660 mm (h×w×l) containing two natural strip lights for illumination (T5 Strip Light, 24 W, 6400 K, 1200 lumen, 58 cm length, Nelson Garden). Reference samples receiving no illumination (0 hours) were wrapped in light protective foil before illumination and showed an average of 29 ppt 3-MBT. All samples were illuminated for 4 hours and after illumination, the light was switched off and the samples were immediately wrapped in tin foil and analyzed for 3-MBT content according to the method described in example 5. The result of illuminated samples is shown in FIG. 7. It's clear that increasing the enzyme concentration from 2 to 24 μM of MOXRcaE and MOXRcaB lead to a substantial decrease in the developed 3-MBT from 134 to 90 ppt, corresponding to a relative decrease compared to the controls of 8.7% (2 μM enzyme) to 37.3% (24 Reference samples showed approximately similar values with an average of 141 ppt 3-MBT after the 4 hours of illumination. The quantified concentration of riboflavin in the samples before illumination is shown in FIG. 8. Increasing the concentration of enzyme from 2, 12 to 24 μM resulted in 351, 129 and 14 μg/L riboflavin before illumination respectively. The controls samples showed approximately similar values with an average of 356.6 μg/L. Thus the applied enzyme enabled riboflavin degradation in the beer that resulted in substantial reduction of 3-MBT off-flavor formation from illumination.

### Example 10—Use of BluB Enzyme for Riboflavin Degradation in Beer

The BluB enzyme action would be conversion of riboflavin into DMB, a natural benzimidazole derivative with no expected photosensitize properties. In beer photoexcited riboflavin induces cleavage of isohumulones to a 4-methylpent-3-enoyl radical, which undergoes decarbonylation to a 3-methylbut-2-enyl radical. Thus, BluB facilitated degradation of riboflavin into DMB could remove the photosensitive properties of beer and generate a light-stable beer with no or low 3-MBT formation.

### Example 11—Use of Riboflavinhydrolase or BluB Enzyme for Riboflavin Degradation in Milk-Based Dairy Products

The most important mechanism for the formation of singlet oxygen in foods is photosensitized generation by light. Riboflavin, is present in high concentrations in milk where it may act as potent photosensitizers and lead to generation of unwanted off-flavors. This may be in products such as e.g. milk, yogurt, fermented milk products and ice-cream. The use of riboflavinase or BluB enzymes in milk may convert riboflavin into degradation products with low/no photosensitizing properties such as, lumichrome, DMB or similar. Thus, riboflavinase or BluB facilitated degradation of riboflavin may generate light-stable milk-based dairy products.

### Example 12—Use of Riboflavinhydrolase or BluB Enzyme for Riboflavin Degradation in Vegetable Oil

The most important mechanism for the formation of singlet oxygen in foods is photosensitized generation by light. Riboflavin, is present in vegetable oils where it may act as potent photosensitizers and lead to generation of unwanted off-flavors. This may be in oils such as e.g. olive oil, soy bean oil, coconut oil among others. The use of riboflavinase or BluB enzymes in vegetable oils may convert riboflavin into degradation products with low/no photosensitizing properties such as, lumichrome, DMB or similar. Thus, a riboflavinase or BluB facilitated degradation of riboflavin may generate an oil with improved light stability.

SEQ ID NO: 1 sets forth the nucleotide sequence of
full-length SmeBluB1 gene identified from NCBI
database:
ATGCTGCCTGACCCGAACGGCTGCCTTACGGCTGCCGGAGCTTTTTCGTC
GGACGAGCGCGCCGCCGTCTATCGTGCCATTGAGACGCGTCGCGACGTGC
GCGACGAGTTCCTGCCCGAACCATTGTCCGAGGAACTGATCGCCCGCCTG
CTCGGTGCGGCGCACCAGGCGCCGTCCGTCGGCTTCATGCAACCCTGGAA
CTTCGTGCTCGTGCGCCAGGACGAGACGCGGGAGAAAGTCTGGCAGGCTT
TCCAGCGCGCCAATGACGAGGCCGCAGAGATGTTTTCCGGCGAAAGGCAA
GCGAAGTACCGGTCGCTGAAGCTCGAAGGCATTCGCAAGGCGCCGCTCAG
CATTTGCGTGACCTGCGACCGGACGCGCGGCGGAGCGGTCGTCCTGGGCC
GCACCCATAATCCGCAGATGGATCTGTACTCGACCGTTTGCGCCGTCCAG
AACCTCTGGCTCGCCGCGCGGGCGGAGGGCGTGGGCGTCGGCTGGGTCAG
CATTTTCCACGAGAGCGAGATCAAGGCTATCCTCGGAATCCCGGATCATG
TCGAAATCGTCGCCTGGCTGTGCCTCGGTTTCGTCGACAGGCTGTATCAA
GAGCCGGAACTCGCCGCGAAGGGCTGGCGACAGCGCTTGCCGCTCGAAGA
TCTCGTTTTCGAGGAAGGCTGGGGCGTCCGTTAG
SEQ ID NO: 2 sets forth the amino acid sequence of
SmeBluB1:
MLPDPNGCLTAAGAFSSDERAAVYRAIETRRDVRDEFLPEPLSEELIARL
LGAAHQAPSVGFMQPWNFVLVRQDETREKVWQAFQRANDEAAEMFSGERQ
AKYRSLKLEGIRKAPLSICVTCDRTRGGAVVLGRTHNPQMDLYSTVCAVQ
NLWLAARAEGVGVGWVSIFHESEIKAILGIPDHVEIVAWLCLGFVDRLYQ
EPELAAKGWRQRLPLEDLVFEEGWGVR
SEQ ID NO: 3 sets forth the nucleotide sequence of
full-length PspBluB2 gene identified from NCBI
database.
ATGTTTACAGAAGAAGAAAAGGACGGTTTGTACAAGAGCATTTATACAAG
ACGGGATGTACGAACATTTTTATCGGATCCTATCCCAGAAGAAACAATAA
TGAAATTGCTAAATGCAGCGCACCATGGTCCTTCCGTTGGATTTATGCAA
CCCTGGAATTTTATAATCATTTCTACGGAAAAAGTGAAAGAGCGTTTGGC
TTGGGCAGCAGATAAAGAGAGACGAGCTTTAGCCATTCATTATGAAGATA
CACGTCAAGATGAATTTCTAAATCTCAAGATTGAAGGGATTAAGCAGGCA
CCAATTACGATTTGCGTTACTTGTGATCCAACACGCGGCGGTTCTCATGT
GCTAGGAAGAAATTCGATACCAGAAACCGATATCATGTCCGTTGCTTGCG
CGATTCAAAACATGTGGCTAGCGGCATGTGCAGAGGGATTAGCGATGGGC
TGGGTAAGCTTCTATAAGAAAAATGATGTGCGAGATATCCTTGGGATACC
TCCTCATATTGATCCAGTAGCATTGCTATCCATTGGATTCACAGAAAATT
ATCCAGAGAAGCCCATTTTGGAGACAGCCAATTGGGAAAAGCGCAGGAGT
CTGAATAATCTAATATTCAGCGAGACTTGGGGAAATCAAAAAGTTGATTA
G
SEQ ID NO: 4 sets forth the amino acid sequence of
PspBluB2
MFTEEEKDGLYKSIYTRRDVRTFLSDPIPEETIMKLLNAAHHGPSVGFMQ
PWNFIIISTEKVKERLAWAADKERRALAIHYEDTRQDEFLNLKIEGIKQA
PITICVTCDPTRGGSHVLGRNSIPETDIMSVACAIQNMWLAACAEGLAMG
WVSFYKKNDVRDILGIPPHIDPVALLSIGFTENYPEKPILETANWEKRRS
LNNLIFSETWGNQKVD
SEQ ID NO: 5 sets forth the nucleotide sequence of
full-length MoxRcaB1 gene identified from NCBI
database.
ATGACGACTGCCGTGACCGACGCCCTGCCGCGCGACCTCGCCCTCCGCCG
GGCCTTCTCGGTGTATCCGACCGGTGTCGTGGCCCTCGCCGCGCACATCG
ACGATCGAGCCGTCGGGATGGCGGTCAACTCCTTCACCTCGATCTCGCTC
GAACCTGCACTCGTCGCGATCAGCGCAGCCCGCACGTCGAAGACCTGGCC
GGTGCTGCGCACGGTGCCCGAGCTCGGCATGAGCGTGCTCGCAGCGCACC
ACGAGCCGCTCAGCCGCTCGCTCTCGGCGCGTGAGGGCGACCGGTTCGGC
GGACACGAGTGGCAGCGCACCGACGGGGGAGCCGTGCTGATCGCGGATGC
GGCGCTCTGGCTCACCTGCCGCCTGCACAGCACGTTCGACGGCGGGGATC
ACGAGATCGCCCTGTACGAGATCGCCGACGTCACGCTCTTCGACGATGTC
GAGCCCCTGGTCTTCCACCAGAGCCGCTACCGGTCCATCGCCGCGCCCGA
GAGCGCGTGA
SEQ ID NO: 6 sets forth the amino acid sequence of
MoxRcaB1. The peptides display homology to the
Microbacterium maritypicum RcaB are shown in bold.
MTTAVTDALPRDLALRRAFSVYPTGVVALAAHIDDRAVGMAVNSFTSISL
EPALVAISAARTSKTWPVLRTVPELGMSVLAAHHEPLSRSLSAREGDRFG
GHEWQRTDGGAVLIADAALWLTCRLHSTFDGGDHEIALYEIADVTLFDDV
EPLVFHQSRYRSIAAPESA
SEQ ID NO: 7 sets forth the nucleotide sequence of
full-length MoxRcaE1 gene identified from NCBI
database.
ATGACCGATCAGAACACCGTCAAGCCGCTGCGCCTCGGCCTCTTCGAGAA
CGCACAGGCGAACGACTCCGGTACCGCGACCTGGCGGCACCCCGACAACG
GGCGCTACCTGTTCGACAAGCTCGAGTACTGGCGCGACACCGCGCGCATG
GTCGAGGACGCCGGGTTCGACTTCCTTTTCCTCGCGGATGCCTGGGGCTG
GGCCGACGTCGCGGGTGAACGCCCCGACATCTGCTCCGTCGAAGGCCTCG
ATCTGCCCCGGCTCGACCCGGCGATCATCCTTGCCGCACTCATCCCCGAG
ACCACGCGCCTGGGGCTCGTCGCCACCGGATCCACGCTGCTCGAGCCGCC
CTACTCGTTCGCGCGCCGCATGGCGACCCTCGACATCCTCTCGGGCGGAC
GCATCGGCTGGAACGTGGTCACCACGGGCACGGCCGACACCGCGGTGCAG
GGCTTCGGTGTGCCGATGGTCGGCCACGACGAGCGCTACCTCATGGCCGA
CGACTTCATGCAGGTCGTCTACAAGCTGTGGGAGCAGGCCTGGGAGGAAG
GCGCCCTGGAGCGCGACAAGGCCGGTCGCTTCGCCGACCCGTCCAAGGTG
CACCGCATCGCGCACGACGGTCCCTATTTCCGCTCGCACGGCTACGGCAA
CACCTCGCGCTCGCCGCAGGGGACGCCGGTGCTGTTCCAGGCCGGGGCAT
CGCCCGCCGGTCGCGAGTTCGGCGGCAAGCACGGCGAGGCGATCTTCGTC
GGCAGCGGTTCGGTCGAACAGCTCAGTGCGCACTCGAGTGCGATCCGCGA
GGAGGCCGTGAAGAACGGTCGCGGGGCCGACGAGGTCAAGATCATGTCGG
CGTTCGCCGCGGTCGTCGGCAGCACCGAGGAGGAGGCCCGGCGCAAGTAC
GCCGAGGTCGCCGACGCGCAGAACCCCGATGTCACGGTCGCCTCCTACGC
ATGGTTCACCGGCCTCGACCTCTCGGCGTACGCGCCGAACACGCCGATGT
CGGAGCTCAGCACCGAACTCTCGCAGACGCAGGTCGCGCGCTTCGCGGAC
AAGACCGTCGGCGATGTGCTGGGGGATTGGCACGCGCACGGCGTCGGAGC
CCGCCCCATCGTCGGCACCCCGGAGCAGGTCGCTGACCGGATGATCGAGC
TCGCCGACGGCGCCGACCTCGACGGGTTCCTGTTCGCCCCCGTCATCCCG
CCGGCCTCGACCGTCGACTTCATCGAGCACGTGCTGCCGATCCTCAAGGA
GCGCGGCGCGATCGCGGAGCCCTCGTCCGAGCCGCAGTCGCTGCGCGAGC
GCCTGATCGGCACGCCCACGCCGGCGCTCGCAGAGTCGCACACGGGCTCG
CAGTACCGGCGGACGATGTCGCGTGTCTGA
SEQ ID NO: 8 sets forth the amino acid sequence of
MoxRcaE1. The peptides display homology to the
Microbacterium maritypicum RcaE are shown in bold.
MTDQNTVKPLRLGLFENAQANDSGTATWRHPDNGRYLFDKLEYWRDTARM
VEDAGFDFLFLADAWGWADVAGERPDICSVEGLDLPRLDPAIILAALIPE
TTRLGLVATGSTLLEPPYSFARRMATLDILSGGRIGWNVVTTGTADTAVQ
GFGVPMVGHDERYLMADDFMQVVYKLWEQAWEEGALERDKAGRFADPSKV
HRIAHDGPYFRSHGYGNTSRSPQGTPVLFQAGASPAGREFGGKHGEAIFV
GSGSVEQLSAHSSAIREEAVKNGRGADEVKIMSAFAAVVGSTEEEARRKY
AEVADAQNPDVTVASYAWFTGLDLSAYAPNTPMSELSTELSQTQVARFAD
KTVGDVLGDWHAHGVGARPIVGTPEQVADRMIELADGADLDGFLFAPVIP
PASTVDFIEHVLPILKERGAIAEPSSEPQSLRERLIGTPTPALAESHTGS
QYRRTMSRV
SEQ ID NO: 9 sets forth the nucleotide sequence of
full-length MoxRcaB2 gene identified from NCBI
database.
ATGACGACTGCCGTGACTGACGCCTTGCCCCGCGACCTCGCCCTCCGCCG
GGCCTTCTCGGTGTACCCGACCGGTGTCGTGGCGCTCGCCGCCCACGTCG
ACGACCGTGCGGTCGGGATGGCGGTCAACTCCTTCACCTCGATCTCGCTC
GAACCCGCGCTGGTCGCCATCAGTGCGGCCCGCACCTCGAAGACCTGGCC
GGTGCTGCGCGCAGTGCCCGAGCTCGGCATGAGCGTGCTCGCAGCGCACC
ATGAGCCCCTCAGCCGATCGCTCTCGGCGCGCGAGGGCGATCGTTTCGGC
GGGCACGAGTGGCAGCGCACCGAGGGTGGCGCTGTGCTCATCGCCGACGC
GGCCCTGTGGCTCACCTGCCGTCTGCACAGCACCTTCGATGGCGGCGACC
ACGAGGTCGCGCTGTACGAGATCGCTGACGTCACCCTGTTCGACGACGTC
GAGCCGCTGGTCTTCCACCAGAGCCGTTACCGCTCCATCGCCGCTCCCGA
GAGCGCGTGA
SEQ ID NO: 10 sets forth the amino acid sequence of
MoxRcaB2.
MTTAVTDALPRDLALRRAFSVYPTGVVALAAHVDDRAVGMAVNSFTSISL
EPALVAISAARTSKTWPVLRAVPELGMSVLAAHHEPLSRSLSAREGDRFG
GHEWQRTEGGAVLIADAALWLTCRLHSTFDGGDHEVALYEIADVTLFDDV
EPLVFHQSRYRSIAAPESA
SEQ ID NO: 11 sets forth the nucleotide sequence of
full-length MoxRcaE2 gene identified from NCBI
database.
ATGACCGATCAGAACACCGTCAAGCAGCTGCGTCTCGGCCTCTTCGAGAA
CGCTCAGGCGAACGACTCCGGCACCGCGACCTGGCGTCACCCCGACAACG
GGCGTTACCTGTTCGACAAGCTCGACTACTGGCGCGACACGGCGCGCATG
GTCGAGGACGCCGGGTTCGACTTCCTGTTCCTCGCGGATGCCTGGGGCTG
GGCCGACGTCGCGGGCGAACGCCCCGACATCTGCTCCGTCGAAGGCCTCG
ACCTGCCGCGTCTCGACCCCGCGATCATCCTCGCAGCCCTCATCCCGGAG
ACCACGCGGTTGGGTCTCGTCGCCACGGGATCCACGCTGCTCGAGCCGCC
CTATTCCTTCGCCCGTCGCATGGCCACGCTCGACATCCTCTCCGGCGGTC
GCATCGGCTGGAACGTGGTCACCACCGGGACCGCCGACACCGCGGTGCAG
GGCTTCGGGGTGCCGATGGTCGGGCACGACGAGCGCTACCTCATGGCCGA
CGACTTCATGCAGGTCGTCTACAAGCTGTGGGAGCAGGCGTGGGACGAGG
GAGCCCTGGAGCGCGACAAGTCCGGCCGCTTCGCCGACCCGTCCAAGGTG
CACCGCATCGCGCACGACGGCCCCTACTTCCGCTCGCACGGCTACGGCAA
CACGGCGCGCTCGCCGCAGGGCACCCCCGTGCTGTTCCAGGCCGGAGCAT
CGCCCGCCGGTCGTGAGTTCGGCGGCAAGCACGGCGAGGCGATCTTCGTG
GGCAGCGGTTCGGTCGAACAGCTCCGTGCGCACTCGAGCGCCATCCGCGA
GGAGGCCATCAAGAACGGTCGCGGGGCCGACGAGGTCAAGATCATGTCGG
CGTTCGCGGCGGTCGTCGGCAGCACCGAGGAGGAGGCCCGGCGCAAGTAC
GCCGAGGTGTCCGATGCGCAGAACCCCGACGTCACCGTCGCCTCCTACGC
CTGGTTCACCGGGCTCGACCTCTCGGCGTACGCACCGGACACACCGATGT
CGGAGCTCAGCACCGAGCTCTCGCAGACGCAGGTCGCCCGCTTCGCCGAC
AAGACCGTCGGCGATGTGCTGGGAGACTGGCATGCGCACGGCGTCGGAGC
CCGCCCGATCGTGGGCACTCCGGAGCAGGTAGCCGACCGGATGATCCAGC
TCGCCGACGGCGCCGACCTCGACGGGTTCCTGTTCGCCCCCGTCATCCCG
CCCGCCTCGACCGTCGACTTCATCGAGCACGTGCTGCCGATCCTCAAGGA
GCGCGGCGCGATCGCGGAGCCCTCGACGGAGCCGCAGTCGCTGCGTGAGC
GCCTGATCGGCACGCCGACACCTGCGCTCGCCGAGTCGCACACCGGCTCG
CAGTTCCGGCGGACGATGTCGCGTGTCTGA
SEQ ID NO: 12 sets forth the amino acid sequence of
MoxRcaE2.
MTDQNTVKQLRLGLFENAQANDSGTATWRHPDNGRYLFDKLDYWRDTARM
VEDAGFDFLFLADAWGWADVAGERPDICSVEGLDLPRLDPAIILAALIPE
TTRLGLVATGSTLLEPPYSFARRMATLDILSGGRIGWNVVTTGTADTAVQ
GFGVPMVGHDERYLMADDFMQVVYKLWEQAWDEGALERDKSGRFADPSKV
HRIAHDGPYFRSHGYGNTARSPQGTPVLFQAGASPAGREFGGKHGEAIFV
GSGSVEQLRAHSSAIREEAIKNGRGADEVKIMSAFAAVVGSTEEEARRKY
AEVSDAQNPDVTVASYAWFTGLDLSAYAPDTPMSELSTELSQTQVARFAD
KTVGDVLGDWHAHGVGARPIVGTPEQVADRMIQLADGADLDGFLFAPVIP
PASTVDFIEHVLPILKERGAIAEPSTEPQSLRERLIGTPTPALAESHTGS
QFRRTMSRV
SEQ ID NO: 13 sets forth the nucleotide sequence of
the synthesized PspBluB2 gene in plasmid p3JM-
PspBluB2.
ATGTTTACAGAAGAAGAAAAAGATGGACTGTACAAATCTATATACACAAG
AAGAGATGTTAGAACATTTCTGAGCGATCCGATTCCGGAAGAAACAATTA
TGAAACTGCTTAATGCAGCACATCATGGACCGTCAGTTGGCTTTATGCAA
CCGTGGAATTTTATTATTATTTCAACAGAAAAAGTTAAAGAAAGACTTGC
ATGGGCAGCAGATAAAGAAAGAAGAGCACTTGCAATTCATTATGAAGATA
CAAGACAAGATGAATTTCTTAATCTTAAAATTGAAGGCATTAAACAAGCA
CCGATTACAATTTGCGTTACATGCGATCCGACAAGGGGCGGCTCTCATGT
TCTTGGCAGAAATAGCATTCCGGAAACAGATATTATGAGCGTTGCATGCG
CAATTCAAAATATGTGGTTAGCAGCATGCGCAGAAGGCCTGGCAATGGGC
TGGGTTAGCTTTTACAAGAAAAATGATGTTAGAGATATTCTTGGCATTCC
GCCGCATATTGATCCGGTTGCACTGCTGTCTATTGGCTTTACAGAAAATT
ATCCGGAAAAACCGATTCTGGAAACAGCAAATTGGGAAAAAAGAAGATCA
CTGAATAACCTGATTTTTAGCGAAACATGGGGCAATCAAAAAGTTGAT
SEQ ID NO: 14 sets forth the nucleotide sequence of
the synthesized MoxRcaE1 gene in plasmid p3JM-
MoxRcaE1.
ATGACGGATCAAAATACAGTTAAACCGCTGCGCCTGGGCCTTTTTGAAAA
TGCACAAGCAAATGATAGCGGCACGGCAACGTGGAGACATCCGGATAATG
GCCGCTATCTGTTTGATAAACTTGAATATTGGAGAGATACAGCAAGAATG
GTTGAAGATGCAGGATTTGATTTTCTGTTTCTGGCAGATGCATGGGGCTG
GGCAGATGTTGCAGGCGAAAGACCGGATATCTGCTCAGTTGAAGGCCTTG
ATCTACCGAGACTTGATCCGGCAATCATCTTAGCAGCACTTATCCCTGAA
ACGACACGCCTGGGACTGGTTGCAACGGGCTCTACACTGCTTGAACCTCC
GTATAGCTTTGCACGCCGCATGGCAACGTTAGATATTCTGAGCGGAGGAC
GCATCGGCTGGAATGTTGTTACGACAGGAACAGCAGATACGGCAGTTCAA
GGCTTTGGCGTTCCTATGGTTGGCCATGATGAACGCTACCTAATGGCAGA
TGATTTTATGCAAGTTGTTTACAAGCTGTGGGAACAAGCATGGGAAGAAG
GCGCATTAGAACGCGATAAAGCAGGCCGCTTTGCAGATCCGTCTAAAGTT
CATAGAATCGCACATGATGGACCGTATTTTCGCTCTCATGGCTATGGAAA
TACGTCTCGCTCTCCTCAAGGAACACCGGTTCTTTTTCAAGCAGGCGCAA
GCCCGGCAGGCCGCGAATTTGGAGGAAAACATGGAGAAGCAATCTTTGTT
GGCTCAGGATCAGTTGAACAACTGAGCGCACATTCTAGCGCAATTCGCGA
AGAAGCAGTTAAAAACGGACGCGGCGCAGATGAAGTTAAAATTATGTCAG
CATTTGCAGCAGTTGTTGGCTCTACGGAAGAAGAAGCACGCCGCAAATAT
GCAGAAGTTGCAGATGCACAAAATCCTGATGTTACAGTTGCATCCTATGC
ATGGTTTACAGGACTGGATCTGTCAGCGTATGCACCTAATACACCTATGA
GCGAACTGTCTACGGAATTAAGCCAAACACAAGTTGCACGCTTTGCAGAT
AAAACGGTTGGCGATGTTTTAGGCGATTGGCATGCACATGGCGTTGGAGC
ACGCCCTATCGTTGGAACACCTGAACAAGTTGCAGATAGAATGATCGAAT
TAGCAGATGGAGCAGATCTTGATGGCTTTCTGTTTGCACCGGTTATTCCT
CCGGCATCTACGGTTGATTTTATCGAACATGTTCTTCCGATCCTTAAAGA
AAGAGGCGCAATCGCAGAACCGAGCTCTGAACCTCAATCACTTCGCGAAA
GACTTATCGGAACGCCGACACCTGCATTAGCAGAAAGCCATACGGGCTCA
CAATATAGACGCACGATGTCTCGCGTTTAA
SEQ ID NO: 15 sets forth the nucleotide sequence of
the synthesized MoxRcaE2 gene in plasmid p3JM-
MoxRcaE2.
ATGACGGATCAAAATACAGTTAAACAACTTAGACTGGGACTTTTTGAAAA
TGCACAAGCAAATGATTCAGGAACGGCAACGTGGAGACATCCGGATAATG
GCCGCTACCTATTTGATAAACTTGATTATTGGAGAGATACAGCAAGAATG
GTTGAAGATGCAGGCTTTGATTTTCTGTTTCTGGCAGATGCATGGGGCTG
GGCAGATGTTGCAGGCGAAAGACCTGATATCTGCTCAGTTGAAGGCCTTG
ATCTGCCTCGCCTTGATCCGGCAATCATCTTAGCAGCATTAATTCCTGAA
ACGACGAGACTGGGACTGGTTGCAACGGGCTCTACGTTACTTGAACCTCC
GTATAGCTTTGCACGCCGCATGGCAACGTTAGATATCTTAAGCGGAGGCC
GCATCGGCTGGAATGTTGTTACGACAGGAACGGCAGATACAGCAGTTCAA
GGCTTTGGAGTTCCTATGGTTGGACATGATGAACGCTATTTGATGGCAGA
TGATTTTATGCAAGTTGTTTACAAGCTGTGGGAACAAGCATGGGATGAAG
GCGCATTAGAAAGAGATAAATCAGGCCGCTTTGCAGATCCGTCTAAAGTT
CATAGAATCGCACATGATGGCCCGTATTTTCGCTCTCATGGCTATGGAAA
TACAGCACGCTCTCCTCAAGGAACACCGGTTCTGTTTCAAGCGGGAGCAT
CTCCTGCAGGACGCGAATTTGGAGGAAAACATGGCGAAGCAATCTTTGTT
GGATCAGGAAGCGTTGAACAACTGAGAGCACATTCTAGCGCAATTCGCGA
AGAAGCAATTAAAAACGGTCGCGGAGCAGATGAAGTTAAAATTATGAGCG
CATTTGCAGCAGTTGTTGGCTCTACGGAAGAAGAAGCACGCCGCAAATAT
GCAGAAGTTAGCGATGCACAAAATCCGGATGTTACAGTTGCATCTTATGC
ATGGTTTACGGGCCTGGATCTGAGCGCATACGCACCGGATACACCTATGT
CTGAACTGTCTACGGAACTGTCACAAACACAAGTTGCACGCTTTGCAGAT
AAAACGGTTGGCGATGTTTTAGGCGATTGGCATGCACATGGCGTTGGCGC
ACGCCCTATCGTTGGAACACCTGAACAAGTTGCAGATAGAATGATTCAAT
TAGCAGATGGAGCAGATCTTGATGGCTTTCTGTTTGCACCGGTTATCCCT
CCGGCATCTACGGTTGATTTTATCGAACATGTTCTTCCGATCCTTAAAGA
AAGAGGCGCAATCGCAGAACCGTCTACAGAACCTCAATCACTTCGCGAAA
GACTTATCGGCACACCGACACCGGCACTGGCAGAATCTCATACGGGCTCT
CAATTTCGCCGCACGATGTCACGCGTT
SEQ ID NO: 16 sets forth the nucleotide sequence of
the synthesized SmeBluB1 gene in plasmid pET-28b-
SmeBluB1.
ATGTTACCCGATCCGAATGGCTGCCTTACAGCAGCAGGCGCATTTTCTAG
CGATGAAAGAGCAGCAGTTTATAGAGCAATTGAAACAAGAAGAGATGTTA
GAGATGAATTTCTTCCGGAACCGCTGAGCGAAGAACTGATTGCAAGATTA
TTAGGCGCAGCACATCAAGCACCGTCAGTTGGCTTTATGCAACCGTGGAA
TTTTGTTCTGGTTAGACAAGATGAAACAAGAGAAAAAGTTTGGCAAGCAT
TTCAAAGAGCAAATGATGAAGCAGCAGAAATGTTTAGCGGCGAAAGACAA
GCAAAATATAGATCACTTAAACTGGAAGGCATTAGAAAAGCACCGCTGTC
TATTTGCGTTACATGCGATAGAACACGAGGGGGCGCAGTTGTTCTTGGCA
GAACACATAATCCGCAGATGGATCTGTATTCTACAGTTTGCGCAGTTCAA
AATCTGTGGCTTGCAGCAAGAGCAGAGGGAGTTGGCGTTGGATGGGTTTC
TATTTTTCATGAATCAGAAATTAAAGCAATTCTGGGCATTCCGGATCATG
TTGAAATTGTTGCATGGTTATGCCTGGGCTTTGTTGATAGACTGTATCAA
GAACCGGAACTTGCAGCAAAAGGATGGAGACAAAGACTGCCGCTTGAAGA
TCTGGTTTTTGAAGAAGGCTGGGGCGTTAGATAA
SEQ ID NO: 17 sets forth the nucleotide sequence of
the synthesized MoxRcaB1 gene in plasmid pET-28b-
MoxRcaB1.
ATGACAACGGCAGTTACGGATGCACTTCCTAGAGATCTTGCATTAAGACG
CGCCTTCAGCGTTTATCCGACGGGAGTTGTTGCACTGGCAGCACATATCG
ATGATAGAGCAGTTGGAATGGCAGTTAATAGCTTTACGAGCATCTCACTT
GAACCGGCATTAGTTGCAATCTCAGCAGCAAGAACGTCTAAAACGTGGCC
GGTTCTGAGAACGGTTCCGGAATTAGGAATGTCTGTTCTGGCAGCACATC
ATGAACCTCTGTCAAGAAGCCTGAGCGCACGCGAAGGCGATAGATTTGGA
GGACATGAATGGCAAAGAACAGATGGAGGCGCAGTTCTTATTGCAGATGC
AGCACTGTGGCTTACATGCCGCCTTCATTCTACGTTTGATGGAGGCGATC
ATGAAATTGCACTGTATGAAATCGCAGATGTTACGTTATTTGATGATGTT
GAACCGCTGGTTTTTCATCAATCACGCTATCGCTCTATCGCAGCACCTGA
AAGCGCATAA
SEQ ID NO: 18 sets forth the nucleotide sequence of
the synthesized MoxRcaB2 gene in plasmid pET-28b-
MoxRcaB2.
ATGACGACGGCAGTTACGGATGCATTACCTAGAGATCTGGCATTAAGACG
CGCCTTCAGCGTTTATCCGACGGGAGTTGTTGCACTGGCAGCACATGTTG
ATGATAGAGCAGTTGGAATGGCAGTTAATAGCTTTACATCTATCTCTCTG
GAACCGGCACTGGTTGCAATTAGCGCAGCAAGAACGTCTAAAACGTGGCC
GGTTCTGAGAGCAGTTCCGGAATTAGGAATGTCTGTTCTTGCAGCACATC
ATGAACCGCTGTCACGCTCACTTTCAGCACGCGAAGGCGATAGATTTGGA
GGACATGAATGGCAAAGAACAGAGGGTGGCGCAGTTCTTATTGCAGATGC
AGCACTGTGGCTGACATGCCGCCTTCATTCTACGTTTGATGGAGGCGATC
ATGAAGTTGCATTATATGAAATCGCAGATGTTACGTTATTTGATGATGTT
GAACCTCTTGTTTTTCATCAAAGCCGCTATCGCTCAATCGCAGCACCTGA
AAGCGCATAA
SEQ ID NO: 19 sets forth the amino acid sequence of
SmeBluB1 expressed from plasmid pET-28b-SmeBluB1.
The thrombin cleavage peptide was showed in bold
and the 6x His-tag was showed in italics.
MGSSHHHHHHSSGLVPRGSHMLPDPNGCLTAAGAFSSDERAAVYRAIETR
RDVRDEFLPEPLSEELIARLLGAAHQAPSVGFMQPWNFVLVRQDETREKV
WQAFQRANDEAAEMFSGERQAKYRSLKLEGIRKAPLSICVTCDRTRGGAV
VLGRTHNPQMDLYSTVCAVQNLWLAARAEGVGVGWVSIFHESEIKAILGI
PDHVEIVAWLCLGFVDRLYQEPELAAKGWRQRLPLEDLVFEEGWGVR
SEQ ID NO: 20 sets forth the amino acid sequence of
MoxRcaB1 expressed from plasmid pET-28b-MoxRcaB1.
The thrombin cleavage peptide was showed in bold
and the 6x His-tag was showed in italics.
MGSSHHHHHHSSGLVPRGSHMTTAVTDALPRDLALRRAFSVYPTGVVALA
AHIDDRAVGMAVNSFTSISLEPALVAISAARTSKTWPVLRTVPELGMSVL
AAHHEPLSRSLSAREGDRFGGHEWQRTDGGAVLIADAALWLTCRLHSTFD
GGDHEIALYEIADVTLFDDVEPLVFHQSRYRSIAAPESA
SEQ ID NO. 21 sets forth the amino acid sequence of
MoxRcaB2 expressed from plasmid pET-28b-MoxRcaB2.
The thrombin cleavage peptide was showed in bold
and the 6x His-tag was showed in italics.
MGSSHHHHHHSSGLVPRGSHMTTAVTDALPRDLALRRAFSVYPTGVVALA
AHVDDRAVGMAVNSFTSISLEPALVAISAARTSKTWPVLRAVPELGMSVL
AAHHEPLSRSLSAREGDRFGGHEWQRTEGGAVLIADAALWLTCRLHSTFD
GGDHEVALYEIADVTLFDDVEPLVFHQSRYRSIAAPESA

What is claimed is:

1. A method for the inhibition of formation of 3MBT (3-methylbut-ene-thiol) in a malt beverage comprising adding to the malt beverage an effective amount of a riboflavinase.

2. A method according to claim 1 wherein the riboflavinase is a riboflavin hydrolase.

3. A method according to claim 2 wherein the riboflavin hydrolase comprises an enzyme having at least 80% sequence identity to MOXRcaE1 (SEQ ID NO:8) or an active fragment thereof or MOXRcaE2 (SEQ ID NO:12) or an active fragment thereof.

4. A method according to claim 3 wherein the riboflavin hydrolase comprises an enzyme having at least 80% sequence identity to MOXRcaE1 or an active fragment thereof.

5. A method according to claim 4 wherein the riboflavin hydrolase comprises an enzyme having at least 90% sequence identity to MOXRcaE1 or an active fragment thereof.

6. A method according to claim 5 wherein the riboflavin hydrolase comprises an enzyme having at least 95% sequence identity to MOXRcaE1 or an active fragment thereof.

7. A method according to claim 6 wherein the riboflavin hydrolase comprises an enzyme having at least 99% sequence identity to MOXRcaE1 or an active fragment thereof.

8. A method according to claim 7 wherein said riboflavin hydrolase comprises MOXRcaE1 or an active fragment thereof.

9. A method according to claim 3 wherein said riboflavin hydrolase comprises an enzyme having at least 80% sequence identity to MOXRcaE2 or an active fragment thereof.

10. A method according to claim 9 wherein said riboflavin hydrolase comprises an enzyme having at least 90% sequence identity to MOXRcaE2 or an active fragment thereof.

11. A method according to claim 10 wherein said riboflavin hydrolase comprises an enzyme having at least 95% sequence identity to MOXRcaE2 or an active fragment thereof.

12. A method according to claim 11 wherein said riboflavin hydrolase comprises an enzyme having at least 99% sequence identity to MOXRcaE2 or an active fragment thereof.

13. A method according to claim 12 wherein said riboflavin hydrolase comprises MOXRcaE2 or an active fragment thereof.

14. A method according to claim 2 further comprising adding an effective amount of a second riboflavinase enzyme.

15. A method according to claim 14 wherein said second riboflavinase comprises a riboflavin reductase.

16. A method according to claim 15 wherein the riboflavin reductase is selected from the group consisting of an enzyme having at least 80% sequence identity with MOXRcaB1 (SEQ ID NO:6) or an active fragment thereof or MOXRcaB2 (SEQ ID NO:10) or an active fragment thereof.

17. A method according to claim 16 wherein the riboflavin reductase comprises an enzyme having at least 80% amino acid sequence identity to MOXRcaB1 or an active fragment thereof.

18. A method according to claim 17 wherein said riboflavin reductase comprises an enzyme having at least 90% amino acid sequence identity to MOXRcaB1 or an active fragment thereof.

19. A method according to claim 18 wherein said riboflavin reductase comprises an enzyme having at least 95% amino acid sequence identity to MOXRcaB1 or an active fragment thereof.

20. A method according to claim 19 wherein said riboflavin reductase comprises an enzyme having at least 99% amino acid sequence identity to MOXRcaB1 or an active fragment thereof.

21. A method according to claim 20 wherein said riboflavin reductase comprises MOXRcaB1 or an active fragment thereof.

22. A method according to claim 16 wherein the riboflavin reductase comprises an enzyme having at least 80% amino acid sequence identity to MOXRcaB2 or an active fragment thereof.

23. A method according to claim 17 wherein said riboflavin reductase comprises an enzyme having at least 90% amino acid sequence identity to MOXRcaB2 or an active fragment thereof.

24. A method according to claim 18 wherein said riboflavin reductase comprises an enzyme having at least 95% amino acid sequence identity to MOXRcaB2 or an active fragment thereof.

25. A method according to claim 19 wherein said riboflavin reductase comprises an enzyme having at least 99% amino acid sequence identity to MOXRcaB2 or an active fragment thereof.

26. A method according to claim 20 wherein said riboflavin reductase is MOXRcaB2 or an active fragment thereof.

27. A method according to any of the preceding claims in which the malt beverage is selected from the group consisting of a beer, ale, dry beer, near beer, light beer, low alcohol beer, low calorie beer, porter, bock beer, stout, malt liquor, and non-alcoholic malt liquor.

28. A method according to claim 27 in which the malt beverage comprises a beer.

29. A malt beverage comprising an effective amount of a riboflavinase.

30. A malt beverage according to claim 29 wherein the riboflavinase comprises a riboflavin hydrolase.

31. A malt beverage according to claim 30 wherein the riboflavin hydrolase is selected from the group consisting of an enzyme having at least 80% amino acid sequence identity to MOXRcaE1 (SEQ ID NO:8) or an active fragment thereof or MOXRcaE2 (SEQ ID NO:12) or an active fragment thereof.

32. A malt beverage according to claim 31 wherein said riboflavin hydrolase an enzyme having at least 80% amino acid sequence identity to MOXRcaE1 or an active fragment thereof.

33. A malt beverage according to claim 32 wherein said riboflavin hydrolase comprises an enzyme having at least 90% amino acid sequence identity to MOXRcaE1 or an active fragment thereof.

34. A malt beverage according to claim 33 wherein said riboflavin hydrolase comprises an enzyme having at least 95% amino acid sequence identity to MOXRcaE1 or an active fragment thereof.

35. A malt beverage according to claim 34 wherein said riboflavin hydrolase comprises an enzyme having at least 99% amino acid sequence identity to MOXRcaE1 or an active fragment thereof.

36. A malt beverage according to claim 35 wherein said riboflavin hydrolase comprises MOXRcaE1 or an active fragment thereof.

37. A malt beverage according to claim 31 wherein said riboflavin hydrolase comprises an enzyme having at least 80% sequence identity to MOXRcaE2 or an active fragment thereof.

38. A malt beverage according to claim 37 wherein said riboflavin hydrolase comprises an enzyme having at least 90% sequence identity to MOXRcaE2 or an active fragment thereof.

39. A malt beverage according to claim 38 wherein said riboflavin hydrolase comprises an enzyme having at least 95% sequence identity to MOXRcaE2 or an active fragment thereof.

40. A malt beverage according to claim 39 wherein said riboflavin hydrolase comprises an enzyme having at least 99% amino acid sequence identity to MOXRcaE2 or an active fragment thereof.

41. A malt beverage according to claim 40 wherein said riboflavin hydrolase comprises MOXRcaE2 or an active fragment thereof.

42. A malt beverage according to claim 29 further comprising adding an effective amount of a second riboflavinase enzyme.

43. A malt beverage according to claim 42 wherein said second riboflavinase comprises a riboflavin reductase.

44. A malt beverage according to claim 43 wherein the riboflavin reductase comprises an enzyme having at least 80% amino acid sequence identity to MOXRcaB1 (SEQ ID NO:6) or an active fragment thereof or MOXRcaB2 (SEQ ID NO:10) or an active fragment thereof.

45. A malt beverage according to claim 44 wherein the riboflavin reductase comprises an enzyme having at least 80% amino acid sequence identity to MOXRcaB1 or an active fragment thereof.

46. A malt beverage according to claim 45 wherein said riboflavin reductase comprises an enzyme having at least 90% amino acid sequence identity to MOXRcaB1 or an active fragment thereof.

47. A malt beverage according to claim 46 wherein said riboflavin reductase comprises an enzyme having at least 95% amino acid sequence identity to MOXRcaB1 or an active fragment thereof.

48. A malt beverage according to claim 47 wherein said riboflavin reductase comprises an enzyme having at least 99% amino acid sequence identity to MOXRcaB1 or an active fragment thereof.

49. A malt beverage according to claim 48 wherein said riboflavin reductase comprises MOXRcaB1 or an active fragment thereof.

50. A malt beverage according to claim 44 wherein the riboflavin reductase comprises an enzyme having at least 80% amino acid sequence identity to MOXRcaB2 or an active fragment thereof.

51. A malt beverage according to claim 50 wherein said riboflavin reductase comprises an enzyme having at least 90% amino acid sequence identity to MOXRcaB2 or an active fragment thereof.

52. A malt beverage according to claim 51 wherein said riboflavin reductase comprises an enzyme having at least 95% amino acid sequence identity to MOXRcaB2 or an active fragment thereof.

53. A malt beverage according to claim 52 wherein said riboflavin reductase comprises an enzyme having at least 99% amino acid sequence identity to MOXRcaB2 or an active fragment thereof.

54. A malt beverage according to claim 53 wherein said riboflavin reductase comprises MOXRcaB2 or an active fragment thereof.

55. A malt beverage according to any of the preceding claims in which the malt beverage is selected from the group consisting of a beer, ale, dry beer, near beer, light beer, low alcohol beer, low calorie beer, porter, bock beer, stout, malt liquor, and non-alcoholic malt liquor.

56. A malt beverage according to claim 27 in which the malt beverage is a beer.

57. A method according to claim 1 wherein the riboflavinase is a riboflavin destructase.

58. A method according to claim 57 wherein the riboflavin destructase comprises an enzyme having at least 80% identity to SmeBluB1 (SEQ ID NO:2) or an active fragment thereof or PspBluB1 (SEQ ID NO:4) or an active fragment thereof.

59. A method according to claim 58 wherein the riboflavin destructase comprises an enzyme having at least 80% sequence identity to SmeBluB1 or an active fragment thereof.

60. A method according to claim 59 wherein the riboflavin destructase comprises an enzyme having at least 90% sequence identity to SmeBluB1 or an active fragment thereof.

61. A method according to claim 60 wherein the riboflavin destructase comprises an enzyme having at least 95% sequence identity to SmeBluB1 or an active fragment thereof.

62. A method according to claim 61 wherein the riboflavin destructase comprises an enzyme having at least 99% sequence identity to SmeBluB1 or an active fragment thereof.

63. A method according to claim 62 wherein the riboflavin destructase comprises SmeBluB1 or an active fragment thereof.

64. A method according to claim 58 wherein the riboflavin destructase comprises an enzyme having at least 80% sequence identity to PspBluB1 or an active fragment thereof.

65. A method according to claim 64 wherein the riboflavin destructase comprises an enzyme having at least 90% sequence identity to PspBluB1 or an active fragment thereof.

66. A method according to claim 65 wherein the riboflavin destructase comprises an enzyme having at least 95% sequence identity to PspBluB1 or an active fragment thereof.

67. A method according to claim 66 wherein the riboflavin destructase comprises an enzyme having at least 99% sequence identity to PspBluB1 or an active fragment thereof.

68. A method according to claim 67 wherein the riboflavin destructase comprises PspBluB1 or an active fragment thereof.

69. A method for the inhibition of formation of 3MBT (3-methylbut-ene-thiol) in a food comprising adding to the food an effective amount of a riboflavinase.

70. A method according to claim 69 wherein the food is selected from the group consisting of milk, milk-based dairy product, fermented milk products, ice-cream, vegetable oil, olive oil, soy milk, soy bean oil and oil containing salad dressing.

71. A method according to claim 69 wherein said riboflavinase is selected from the group consisting of a riboflavin hydrolase, a riboflavin reductase and a riboflavin destructase.


SEQUENCE LISTING (21 sequences)

SEQ ID NO: 1 - DNA, 684 bp, Sinorhizobium meliloti
atgctgcctgacccgaacggctgccttacggctgccggagctttttcgtcggacgagcgcgccgccgtctatcgtgccattgagacgcgtcgcgacgtgcgcgacgagttcctgcccgaaccattgtccgaggaactgatcgcccgcctgctcggtgcggcgcaccaggcgccgtccgtcggcttcatgcaaccctggaacttcgtgctcgtgcgccaggacgagacgcgggagaaagtctggcaggctttccagcgcgccaatgacgaggccgcagagatgttttccggcgaaaggcaagcgaagtaccggtcgctgaagctcgaaggcattcgcaaggcgccgctcagcatttgcgtgacctgcgaccggacgcgcggcggagcggtcgtcctgggccgcacccataatccgcagatggatctgtactcgaccgtttgcgccgtccagaacctctggctcgccgcgcgggcggagggcgtgggcgtcggctgggtcagcattttccacgagagcgagatcaaggctatcctcggaatcccggatcatgtcgaaatcgtcgcctggctgtgcctcggtttcgtcgacaggctgtatcaagagccggaactcgccgcgaagggctggcgacagcgcttgccgctcgaagatctcgttttcgaggaaggctggggcgtccgttag

SEQ ID NO: 2 - protein, 227 aa, Sinorhizobium meliloti
MLPDPNGCLTAAGAFSSDERAAVYRAIETRRDVRDEFLPEPLSEELIARLLGAAHQAPSVGFMQPWNFVLVRQDETREKVWQAFQRANDEAAEMFSGERQAKYRSLKLEGIRKAPLSICVTCDRTRGGAVVLGRTHNPQMDLYSTVCAVQNLWLAARAEGVGVGWVSIFHESEIKAILGIPDHVEIVAWLCLGFVDRLYQEPELAAKGWRQRLPLEDLVFEEGWGVR

SEQ ID NO: 3 - DNA, 651 bp, Paenibacillus sp
atgtttacagaagaagaaaaggacggtttgtacaagagcatttatacaagacgggatgtacgaacatttttatcggatcctatcccagaagaaacaataatgaaattgctaaatgcagcgcaccatggtccttccgttggatttatgcaaccctggaattttataatcatttctacggaaaaagtgaaagagcgtttggcttgggcagcagataaagagagacgagctttagccattcattatgaagatacacgtcaagatgaatttctaaatctcaagattgaagggattaagcaggcaccaattacgatttgcgttacttgtgatccaacacgcggcggttctcatgtgctaggaagaaattcgataccagaaaccgatatcatgtccgttgcttgcgcgattcaaaacatgtggctagcggcatgtgcagagggattagcgatgggctgggtaagcttctataagaaaaatgatgtgcgagatatccttgggatacctcctcatattgatccagtagcattgctatccattggattcacagaaaattatccagagaagcccattttggagacagccaattgggaaaagcgcaggagtctgaataatctaatattcagcgagacttggggaaatcaaaaagttgattag

SEQ ID NO: 4 - protein, 216 aa, Paenibacillus sp
MFTEEEKDGLYKSIYTRRDVRTFLSDPIPEETIMKLLNAAHHGPSVGFMQPWNFIIISTEKVKERLAWAADKERRALAIHYEDTRQDEFLNLKIEGIKQAPITICVTCDPTRGGSHVLGRNSIPETDIMSVACAIQNMWLAACAEGLAMGWVSFYKKNDVRDILGIPPHIDPVALLSIGFTENYPEKPILETANWEKRRSLNNLIFSETWGNQKVD

SEQ ID NO: 5 - DNA, 510 bp, Microbacterium oxydans
atgacgactgccgtgaccgacgccctgccgcgcgacctcgccctccgccgggccttctcggtgtatccgaccggtgtcgtggccctcgccgcgcacatcgacgatcgagccgtcgggatggcggtcaactccttcacctcgatctcgctcgaacctgcactcgtcgcgatcagcgcagcccgcacgtcgaagacctggccggtgctgcgcacggtgcccgagctcggcatgagcgtgctcgcagcgcaccacgagccgctcagccgctcgctctcggcgcgtgagggcgaccggttcggcggacacgagtggcagcgcaccgacgggggagccgtgctgatcgcggatgcggcgctctggctcacctgccgcctgcacagcacgttcgacggcggggatcacgagatcgccctgtacgagatcgccgacgtcacgctcttcgacgatgtcgagcccctggtcttccaccagagccgctaccggtccatcgccgcgcccgagagcgcgtga

SEQ ID NO: 6 - protein, 169 aa, Microbacterium oxydans
MTTAVTDALPRDLALRRAFSVYPTGVVALAAHIDDRAVGMAVNSFTSISLEPALVAISAARTSKTWPVLRTVPELGMSVLAAHHEPLSRSLSAREGDRFGGHEWQRTDGGAVLIADAALWLTCRLHSTFDGGDHEIALYEIADVTLFDDVEPLVFHQSRYRSIAAPESA

SEQ ID NO: 7 - DNA, 1380 bp, Microbacterium oxydans
atgaccgatcagaacaccgtcaagccgctgcgcctcggcctcttcgagaacgcacaggcgaacgactccggtaccgcgacctggcggcaccccgacaacgggcgctacctgttcgacaagctcgagtactggcgcgacaccgcgcgcatggtcgaggacgccgggttcgacttccttttcctcgcggatgcctggggctgggccgacgtcgcgggtgaacgccccgacatctgctccgtcgaaggcctcgatctgccccggctcgacccggcgatcatccttgccgcactcatccccgagaccacgcgcctggggctcgtcgccaccggatccacgctgctcgagccgccctactcgttcgcgcgccgcatggcgaccctcgacatcctctcgggcggacgcatcggctggaacgtggtcaccacgggcacggccgacaccgcggtgcagggcttcggtgtgccgatggtcggccacgacgagcgctacctcatggccgacgacttcatgcaggtcgtctacaagctgtgggagcaggcctgggaggaaggcgccctggagcgcgacaaggccggtcgcttcgccgacccgtccaaggtgcaccgcatcgcgcacgacggtccctatttccgctcgcacggctacggcaacacctcgcgctcgccgcaggggacgccggtgctgttccaggccggggcatcgcccgccggtcgcgagttcggcggcaagcacggcgaggcgatcttcgtcggcagcggttcggtcgaacagctcagtgcgcactcgagtgcgatccgcgaggaggccgtgaagaacggtcgcggggccgacgaggtcaagatcatgtcggcgttcgccgcggtcgtcggcagcaccgaggaggaggcccggcgcaagtacgccgaggtcgccgacgcgcagaaccccgatgtcacggtcgcctcctacgcatggttcaccggcctcgacctctcggcgtacgcgccgaacacgccgatgtcggagctcagcaccgaactctcgcagacgcaggtcgcgcgcttcgcggacaagaccgtcggcgatgtgctgggggattggcacgcgcacggcgtcggagcccgccccatcgtcggcaccccggagcaggtcgctgaccggatgatcgagctcgccgacggcgccgacctcgacgggttcctgttcgcccccgtcatcccgccggcctcgaccgtcgacttcatcgagcacgtgctgccgatcctcaaggagcgcggcgcgatcgcggagccctcgtccgagccgcagtcgctgcgcgagcgcctgatcggcacgcccacgccggcgctcgcagagtcgcacacgggctcgcagtaccggcggacgatgtcgcgtgtctga

SEQ ID NO: 8 - protein, 459 aa, Microbacterium oxydans
MTDQNTVKPLRLGLFENAQANDSGTATWRHPDNGRYLFDKLEYWRDTARMVEDAGFDFLFLADAWGWADVAGERPDICSVEGLDLPRLDPAIILAALIPETTRLGLVATGSTLLEPPYSFARRMATLDILSGGRIGWNVVTTGTADTAVQGFGVPMVGHDERYLMADDFMQVVYKLWEQAWEEGALERDKAGRFADPSKVHRIAHDGPYFRSHGYGNTSRSPQGTPVLFQAGASPAGREFGGKHGEAIFVGSGSVEQLSAHSSAIREEAVKNGRGADEVKIMSAFAAVVGSTEEEARRKYAEVADAQNPDVTVASYAWFTGLDLSAYAPNTPMSELSTELSQTQVARFADKTVGDVLGDWHAHGVGARPIVGTPEQVADRMIELADGADLDGFLFAPVIPPASTVDFIEHVLPILKERGAIAEPSSEPQSLRERLIGTPTPALAESHTGSQYRRTMSRV

SEQ ID NO: 9 - DNA, 510 bp, Microbacterium oxydans
atgacgactgccgtgactgacgccttgccccgcgacctcgccctccgccgggccttctcggtgtacccgaccggtgtcgtggcgctcgccgcccacgtcgacgaccgtgcggtcgggatggcggtcaactccttcacctcgatctcgctcgaacccgcgctggtcgccatcagtgcggcccgcacctcgaagacctggccggtgctgcgcgcagtgcccgagctcggcatgagcgtgctcgcagcgcaccatgagcccctcagccgatcgctctcggcgcgcgagggcgatcgtttcggcgggcacgagtggcagcgcaccgagggtggcgctgtgctcatcgccgacgcggccctgtggctcacctgccgtctgcacagcaccttcgatggcggcgaccacgaggtcgcgctgtacgagatcgctgacgtcaccctgttcgacgacgtcgagccgctggtcttccaccagagccgttaccgctccatcgccgctcccgagagcgcgtga

SEQ ID NO: 10 - protein, 169 aa, Microbacterium oxydans
MTTAVTDALPRDLALRRAFSVYPTGVVALAAHVDDRAVGMAVNSFTSISLEPALVAISAARTSKTWPVLRAVPELGMSVLAAHHEPLSRSLSAREGDRFGGHEWQRTEGGAVLIADAALWLTCRLHSTFDGGDHEVALYEIADVTLFDDVEPLVFHQSRYRSIAAPESA

SEQ ID NO: 11 - DNA, 1380 bp, Microbacterium oxydans
atgaccgatcagaacaccgtcaagcagctgcgtctcggcctcttcgagaacgctcaggcgaacgactccggcaccgcgacctggcgtcaccccgacaacgggcgttacctgttcgacaagctcgactactggcgcgacacggcgcgcatggtcgaggacgccgggttcgacttcctgttcctcgcggatgcctggggctgggccgacgtcgcgggcgaacgccccgacatctgctccgtcgaaggcctcgacctgccgcgtctcgaccccgcgatcatcctcgcagccctcatcccggagaccacgcggttgggtctcgtcgccacgggatccacgctgctcgagccgccctattccttcgcccgtcgcatggccacgctcgacatcctctccggcggtcgcatcggctggaacgtggtcaccaccgggaccgccgacaccgcggtgcagggcttcggggtgccgatggtcgggcacgacgagcgctacctcatggccgacgacttcatgcaggtcgtctacaagctgtgggagcaggcgtgggacgagggagccctggagcgcgacaagtccggccgcttcgccgacccgtccaaggtgcaccgcatcgcgcacgacggcccctacttccgctcgcacggctacggcaacacggcgcgctcgccgcagggcacccccgtgctgttccaggccggagcatcgcccgccggtcgtgagttcggcggcaagcacggcgaggcgatcttcgtgggcagcggttcggtcgaacagctccgtgcgcactcgagcgccatccgcgaggaggccatcaagaacggtcgcggggccgacgaggtcaagatcatgtcggcgttcgcggcggtcgtcggcagcaccgaggaggaggcccggcgcaagtacgccgaggtgtccgatgcgcagaaccccgacgtcaccgtcgcctcctacgcctggttcaccgggctcgacctctcggcgtacgcaccggacacaccgatgtcggagctcagcaccgagctctcgcagacgcaggtcgcccgcttcgccgacaagaccgtcggcgatgtgctgggagactggcatgcgcacggcgtcggagcccgcccgatcgtgggcactccggagcaggtagccgaccggatgatccagctcgccgacggcgccgacctcgacgggttcctgttcgcccccgtcatcccgcccgcctcgaccgtcgacttcatcgagcacgtgctgccgatcctcaaggagcgcggcgcgatcgcggagccctcgacggagccgcagtcgctgcgtgagcgcctgatcggcacgccgacacctgcgctcgccgagtcgcacaccggctcgcagttccggcggacgatgtcgcgtgtctga

SEQ ID NO: 12 - protein, 459 aa, Microbacterium oxydans
MTDQNTVKQLRLGLFENAQANDSGTATWRHPDNGRYLFDKLDYWRDTARMVEDAGFDFLFLADAWGWADVAGERPDICSVEGLDLPRLDPAIILAALIPETTRLGLVATGSTLLEPPYSFARRMATLDILSGGRIGWNVVTTGTADTAVQGFGVPMVGHDERYLMADDFMQVVYKLWEQAWDEGALERDKSGRFADPSKVHRIAHDGPYFRSHGYGNTARSPQGTPVLFQAGASPAGREFGGKHGEAIFVGSGSVEQLRAHSSAIREEAIKNGRGADEVKIMSAFAAVVGSTEEEARRKYAEVSDAQNPDVTVASYAWFTGLDLSAYAPDTPMSELSTELSQTQVARFADKTVGDVLGDWHAHGVGARPIVGTPEQVADRMIQLADGADLDGFLFAPVIPPASTVDFIEHVLPILKERGAIAEPSTEPQSLRERLIGTPTPALAESHTGSQFRRTMSRV

SEQ ID NO: 13 - DNA, 648 bp, Artificial Sequence
  synthetic genes
atgtttacagaagaagaaaaagatggactgtacaaatctatatacacaagaagagatgttagaacatttctgagcgatccgattccggaagaaacaattatgaaactgcttaatgcagcacatcatggaccgtcagttggctttatgcaaccgtggaattttattattatttcaacagaaaaagttaaagaaagacttgcatgggcagcagataaagaaagaagagcacttgcaattcattatgaagatacaagacaagatgaatttcttaatcttaaaattgaaggcattaaacaagcaccgattacaatttgcgttacatgcgatccgacaaggggcggctctcatgttcttggcagaaatagcattccggaaacagatattatgagcgttgcatgcgcaattcaaaatatgtggttagcagcatgcgcagaaggcctggcaatgggctgggttagcttttacaagaaaaatgatgttagagatattcttggcattccgccgcatattgatccggttgcactgctgtctattggctttacagaaaattatccggaaaaaccgattctggaaacagcaaattgggaaaaaagaagatcactgaataacctgatttttagcgaaacatggggcaatcaaaaagttgat

SEQ ID NO: 14 - DNA, 1380 bp, Artificial Sequence
  synthetic genes
atgacggatcaaaatacagttaaaccgctgcgcctgggcctttttgaaaatgcacaagcaaatgatagcggcacggcaacgtggagacatccggataatggccgctatctgtttgataaacttgaatattggagagatacagcaagaatggttgaagatgcaggatttgattttctgtttctggcagatgcatggggctgggcagatgttgcaggcgaaagaccggatatctgctcagttgaaggccttgatctaccgagacttgatccggcaatcatcttagcagcacttatccctgaaacgacacgcctgggactggttgcaacgggctctacactgcttgaacctccgtatagctttgcacgccgcatggcaacgttagatattctgagcggaggacgcatcggctggaatgttgttacgacaggaacagcagatacggcagttcaaggctttggcgttcctatggttggccatgatgaacgctacctaatggcagatgattttatgcaagttgtttacaagctgtgggaacaagcatgggaagaaggcgcattagaacgcgataaagcaggccgctttgcagatccgtctaaagttcatagaatcgcacatgatggaccgtattttcgctctcatggctatggaaatacgtctcgctctcctcaaggaacaccggttctttttcaagcaggcgcaagcccggcaggccgcgaatttggaggaaaacatggagaagcaatctttgttggctcaggatcagttgaacaactgagcgcacattctagcgcaattcgcgaagaagcagttaaaaacggacgcggcgcagatgaagttaaaattatgtcagcatttgcagcagttgttggctctacggaagaagaagcacgccgcaaatatgcagaagttgcagatgcacaaaatcctgatgttacagttgcatcctatgcatggtttacaggactggatctgtcagcgtatgcacctaatacacctatgagcgaactgtctacggaattaagccaaacacaagttgcacgctttgcagataaaacggttggcgatgttttaggcgattggcatgcacatggcgttggagcacgccctatcgttggaacacctgaacaagttgcagatagaatgatcgaattagcagatggagcagatcttgatggctttctgtttgcaccggttattcctccggcatctacggttgattttatcgaacatgttcttccgatccttaaagaaagaggcgcaatcgcagaaccgagctctgaacctcaatcacttcgcgaaagacttatcggaacgccgacacctgcattagcagaaagccatacgggctcacaatatagacgcacgatgtctcgcgtttaa

SEQ ID NO: 15 - DNA, 1377 bp, Artificial Sequence
  synthetic genes
atgacggatcaaaatacagttaaacaacttagactgggactttttgaaaatgcacaagcaaatgattcaggaacggcaacgtggagacatccggataatggccgctacctatttgataaacttgattattggagagatacagcaagaatggttgaagatgcaggctttgattttctgtttctggcagatgcatggggctgggcagatgttgcaggcgaaagacctgatatctgctcagttgaaggccttgatctgcctcgccttgatccggcaatcatcttagcagcattaattcctgaaacgacgagactgggactggttgcaacgggctctacgttacttgaacctccgtatagctttgcacgccgcatggcaacgttagatatcttaagcggaggccgcatcggctggaatgttgttacgacaggaacggcagatacagcagttcaaggctttggagttcctatggttggacatgatgaacgctatttgatggcagatgattttatgcaagttgtttacaagctgtgggaacaagcatgggatgaaggcgcattagaaagagataaatcaggccgctttgcagatccgtctaaagttcatagaatcgcacatgatggcccgtattttcgctctcatggctatggaaatacagcacgctctcctcaaggaacaccggttctgtttcaagcgggagcatctcctgcaggacgcgaatttggaggaaaacatggcgaagcaatctttgttggatcaggaagcgttgaacaactgagagcacattctagcgcaattcgcgaagaagcaattaaaaacggtcgcggagcagatgaagttaaaattatgagcgcatttgcagcagttgttggctctacggaagaagaagcacgccgcaaatatgcagaagttagcgatgcacaaaatccggatgttacagttgcatcttatgcatggtttacgggcctggatctgagcgcatacgcaccggatacacctatgtctgaactgtctacggaactgtcacaaacacaagttgcacgctttgcagataaaacggttggcgatgttttaggcgattggcatgcacatggcgttggcgcacgccctatcgttggaacacctgaacaagttgcagatagaatgattcaattagcagatggagcagatcttgatggctttctgtttgcaccggttatccctccggcatctacggttgattttatcgaacatgttcttccgatccttaaagaaagaggcgcaatcgcagaaccgtctacagaacctcaatcacttcgcgaaagacttatcggcacaccgacaccggcactggcagaatctcatacgggctctcaatttcgccgcacgatgtcacgcgtt

SEQ ID NO: 16 - DNA, 684 bp, Artificial Sequence
  synthetic genes
atgttacccgatccgaatggctgccttacagcagcaggcgcattttctagcgatgaaagagcagcagtttatagagcaattgaaacaagaagagatgttagagatgaatttcttccggaaccgctgagcgaagaactgattgcaagattattaggcgcagcacatcaagcaccgtcagttggctttatgcaaccgtggaattttgttctggttagacaagatgaaacaagagaaaaagtttggcaagcatttcaaagagcaaatgatgaagcagcagaaatgtttagcggcgaaagacaagcaaaatatagatcacttaaactggaaggcattagaaaagcaccgctgtctatttgcgttacatgcgatagaacacgagggggcgcagttgttcttggcagaacacataatccgcagatggatctgtattctacagtttgcgcagttcaaaatctgtggcttgcagcaagagcagagggagttggcgttggatgggtttctatttttcatgaatcagaaattaaagcaattctgggcattccggatcatgttgaaattgttgcatggttatgcctgggctttgttgatagactgtatcaagaaccggaacttgcagcaaaaggatggagacaaagactgccgcttgaagatctggtttttgaagaaggctggggcgttagataa

SEQ ID NO: 17 - DNA, 510 bp, Artificial Sequence
  synthetic genes
atgacaacggcagttacggatgcacttcctagagatcttgcattaagacgcgccttcagcgtttatccgacgggagttgttgcactggcagcacatatcgatgatagagcagttggaatggcagttaatagctttacgagcatctcacttgaaccggcattagttgcaatctcagcagcaagaacgtctaaaacgtggccggttctgagaacggttccggaattaggaatgtctgttctggcagcacatcatgaacctctgtcaagaagcctgagcgcacgcgaaggcgatagatttggaggacatgaatggcaaagaacagatggaggcgcagttcttattgcagatgcagcactgtggcttacatgccgccttcattctacgtttgatggaggcgatcatgaaattgcactgtatgaaatcgcagatgttacgttatttgatgatgttgaaccgctggtttttcatcaatcacgctatcgctctatcgcagcacctgaaagcgcataa

SEQ ID NO: 18 - DNA, 510 bp, Artificial Sequence
  synthetic genes
atgacgacggcagttacggatgcattacctagagatctggcattaagacgcgccttcagcgtttatccgacgggagttgttgcactggcagcacatgttgatgatagagcagttggaatggcagttaatagctttacatctatctctctggaaccggcactggttgcaattagcgcagcaagaacgtctaaaacgtggccggttctgagagcagttccggaattaggaatgtctgttcttgcagcacatcatgaaccgctgtcacgctcactttcagcacgcgaaggcgatagatttggaggacatgaatggcaaagaacagagggtggcgcagttcttattgcagatgcagcactgtggctgacatgccgccttcattctacgtttgatggaggcgatcatgaagttgcattatatgaaatcgcagatgttacgttatttgatgatgttgaacctcttgtttttcatcaaagccgctatcgctcaatcgcagcacctgaaagcgcataa

SEQ ID NO: 19 - protein, 247 aa, Artificial Sequence
  recombinant protein
MGSSHHHHHHSSGLVPRGSHMLPDPNGCLTAAGAFSSDERAAVYRAIETRRDVRDEFLPEPLSEELIARLLGAAHQAPSVGFMQPWNFVLVRQDETREKVWQAFQRANDEAAEMFSGERQAKYRSLKLEGIRKAPLSICVTCDRTRGGAVVLGRTHNPQMDLYSTVCAVQNLWLAARAEGVGVGWVSIFHESEIKAILGIPDHVEIVAWLCLGFVDRLYQEPELAAKGWRQRLPLEDLVFEEGWGVR

SEQ ID NO: 20 - protein, 189 aa, Artificial Sequence
  recombinant protein
MGSSHHHHHHSSGLVPRGSHMTTAVTDALPRDLALRRAFSVYPTGVVALAAHIDDRAVGMAVNSFTSISLEPALVAISAARTSKTWPVLRTVPELGMSVLAAHHEPLSRSLSAREGDRFGGHEWQRTDGGAVLIADAALWLTCRLHSTFDGGDHEIALYEIADVTLFDDVEPLVFHQSRYRSIAAPESA

SEQ ID NO: 21 - protein, 189 aa, Artificial Sequence
  recombinant protein
MGSSHHHHHHSSGLVPRGSHMTTAVTDALPRDLALRRAFSVYPTGVVALAAHVDDRAVGMAVNSFTSISLEPALVAISAARTSKTWPVLRAVPELGMSVLAAHHEPLSRSLSAREGDRFGGHEWQRTEGGAVLIADAALWLTCRLHSTFDGGDHEVALYEIADVTLFDDVEPLVFHQSRYRSIAAPESA